(19) United States
(12) Patent Application Publication
(10) Pub. No.: US 2009/0169573 A1
(43) Pub. Date: Jul. 2, 2009

(54) T-Cell Stimulatory Peptides From The Melanoma-Associated Chondroitin Sulfate Proteoglycan And Their Use

(76) Inventors: Erwin Schultz, Amönenburg (DE); Gerold Schuler, Spardorf (DE)

Correspondence Address: Ballard Spahr Andrews & Ingersoll, LLP, SUITE 1000, 999 PEACHTREE STREET, ATLANTA, GA, 30309-3915, US

(21) Appl. No.: 11/665,903

(22) PCT Filed: Oct. 20, 2005
(86) PCT No.: PCT/EP05/55430

§ 371 Date: Apr. 21, 2008

(30) Foreign Application Priority Data
Oct. 20, 2004 (EP) 04105199.6
Dec. 2, 2004 (EP) 04106250.6

(51) Int. Cl. A61K 39/00; C07K 7/00; C12N 15/11; C12N 15/00; C12N 5/06; C12N 15/87; C12Q 1/02; C07K 16/18; A61K 35/12; A61K 31/7088; C12Q 1/68

(52) U.S. Cl. 424/185.1; 530/328; 530/327; 530/326; 530/325; 530/324; 536/23.5; 536/23.4; 435/320.1; 435/325; 435/375; 435/455; 435/29; 530/387.9; 424/93.21; 514/44; 435/6

(57) ABSTRACT

The present invention relates to melanoma-associated chondroitin sulfate proteoglycan (MCSP) epitopes recognized by T cells, especially by CD4⁺ T lymphocytes (short T-cells) and CD8⁺ T cells, on human melanoma cells. In more detail, the present invention relates to novel T-cell stimulatory tumour antigenic peptides corresponding to said epitopes (MCSP peptides); to fusion proteins comprising said MCSP peptides; to the use of said MCSP peptides, fusion proteins or of the full length MCSP protein itself or fragments thereof to induce an immune response, especially a T-cell response; to the use of said MCSP peptides, fusion proteins or full length MCSP protein itself or fragments thereof to prepare immune cells, such as mature dendritic cells (DCs) loaded with anyone of the peptides according to the invention, or peptide-specific T-cell clones, especially CD4⁺ or CD8⁺ T cell clones; to the use of said MCSP peptides, fusion proteins or MCSP itself or fragments thereof for research and development on/of a cancer treatment; to the use of said MCSP peptides, fusion proteins or MCSP itself or fragments thereof for preparing a medicament for inducing a T cell response in a patient, preferably for the treatment of cancer, more preferably for the treatment of melanoma, including cutaneous and ocular melanoma, and other MCSP expressing tumours such as breast cancer, notably lobular breast carcinoma, astrocytoma, glioma, glioblastoma, neuroblastoma, sarcoma and certain types of leukaemia; to the use of said MCSP peptides, fusion proteins or full length MCSP protein or fragments thereof for the preparation of a medicament, and a diagnostic agent for the treatment and prophylaxis as well the diagnosis of an immune response against tumours; and to the use of said peptide-specific T-cell clones for diagnosing or treating cancer.

The present invention relates to melanoma-associated chondroitin sulfate proteoglycan (MCSP) epitopes recognized by T cells, especially by CD4⁺ T lymphocytes (short T-cells) and CD8⁺ T cells, on human melanoma cells. In more detail, the present invention relates to novel T-cell stimulatory tumour antigenic peptides corresponding to said epitopes (MCSP peptides); to fusion proteins comprising said MCSP peptides; to the use of said MCSP peptides, fusion proteins or of the full length MCSP protein itself or fragments thereof to induce an immune response, especially a T-cell response; to the use of said MCSP peptides, fusion proteins or full length MCSP protein itself or fragments thereof to prepare immune cells, such as mature dendritic cells (DCs) loaded with anyone of the peptides according to the invention, or peptide-specific T-cell clones, especially CD4⁺ or CD8⁺ T cell clones; to the use of said MCSP peptides, fusion proteins or MCSP itself or fragments thereof for research and development on/of a cancer treatment; to the use of said MCSP peptides, fusion proteins or MCSP itself or fragments thereof for preparing a medicament for inducing a T cell response in a patient, preferably for the treatment of cancer, more preferably for the treatment of melanoma, including cutaneous and ocular melanoma, and other MCSP expressing tumours such as breast cancer, notably lobular breast carcinoma, astrocytoma, glioma, glioblastoma, neuroblastoma, sarcoma and certain types of leukaemia; to the use of said MCSP peptides, fusion proteins or full length MCSP protein or fragments thereof for the preparation of a medicament, and a diagnostic agent for the treatment and prophylaxis as well the diagnosis of an immune response against tumours; and to the use of said peptide-specific T-cell clones for diagnosing or treating cancer.

### BACKGROUND

During the last two decades there has been considerable interest in the biology and pathophysiology of human malignant melanoma, in particular because of the poor prognosis and increasing incidence of this disease. The fatal nature of metastasizing human cutaneous melanoma which is attributable to poor response to conventional radiation and chemotherapy, has prompted a growing interest in alternative approaches to that disease. One approach was to look for the expression of tumour associated proteins which can be used as targets for immunotherapy. These proteins which are either newly expressed, mutated or overexpressed in tumours, can be utilized for therapy purposes or as diagnostic markers. The human melanoma-associated chondroitin sulfate proteoglycan (MCSP) is a tumour-associated protein. It is uniformly expressed on >90% of human malignant melanoma tissues and cultured cells, but shows only a limited expression pattern in normal tissue (Bumol & Reisfeld, PNAS 79, 1245-1249 (1982); Bumol et al., J. Biol. Chem. 267, 12733-12741 (1984); Bumol et al., Adv. Exp. Med. Biol. 172, 455-470 (1984); Harper et al., J. Biol. Chem. 261, 3600-3606 (1986)). The core protein consists of 2322 amino acids (SEQ ID NO:1), encompassing a large extracellular domain, a hydrophobic transmembrane region, and a relatively short cytoplasmic tail (Pluschke et al., PNAS 93, 9710-9715 (1996) and WO 97/13855). MCSP exists in cells as a unique glycoprotein-proteoglycan complex, with a 250 kDa core glycoprotein to which, via serine residues, the larger than 450 kDa proteoglycan component is attached. Multiple Northern blots with an MCSP-specific probe revealed a strong hybridization signal only with melanoma cells and not with other human cancer cells or a variety of human fetal and adult tissues (Pluschke et al., PNAS 93, 9710-9715 (1996)).

MCSP is a cell-surface antigen that has been implicated in the growth and invasion of melanoma tumours. It was shown that stimulated MCSP recruits an adaptor protein important in tumour cell motility and invasion and participates directly in the signal transduction process crucial for the adhesion and extravasation of tumour cells, and therefore MCSP appears to be relevant for melanoma invasion and metastasis (Iida et al., J. Biol. Chem. 276, 18786-18794 (2001); Burg et al., J. Cell. Physiol. 177, 299-312 (1998); Eisenmann et al., Nat. Cell Biol. 1, 507-513 (1999)). Monoclonal antibodies against MCSP have been available for a long time. They were obtained by immunizing mice with a plasma membrane-enriched fraction from human malignant melanoma cells and subsequent generation of hybridomas (Harper et al., Hybridoma 1, 423-432 (1984); Harper et al., J. Immunol. 132, 2096-2104 (1984)). More recently two antimelanoma MCSP-specific immunoconjugates containing a human single-chain Fv (scFv) targeting domain conjugated to the Fc effector domain of human IgG1 were synthesized. The Fc effector domain of the immunoconjugates binds natural killer (NK) cells and also the C1q protein that initiates the complement cascade, both triggering a powerful cytolytic response against the targeted tumour cells, resulting in the lysis of the melanoma cells (Wang et al., PNAS 96, 1627-1632 (1999)). It is noteworthy that the scFv targeting domains originally were isolated as melanoma-specific clones from a scFv fusion-phage library, derived from the antibody repertoire of a vaccinated melanoma patient (Abdel-Wahab et al., Cancer 80, 401-412 (1997)). However, the promising in vitro results did not hold up in vivo, and the antibody or antibody fragment based approaches towards melanoma have so far not lead to convincing results in the treatment of melanoma (Oldham et al., J. Clin. Oncol. 11:1235-1244 (1984); Abrams et al., in “Monoclonal Antibodies and Cancer Therapy” p. 233 (1985), Reisfeld and Sell eds., New York).

Furthermore, WO 97/13855 utilizes MCSP as an antigen for an active specific immune response for generating a humoral, i.e. antibody response. However, up to now a T-cell inducing property has not been reported for MCSP.

T-cell based approaches towards the treatment of melanoma may hold more promise. A T-cell response requires that T-cells recognize and interact with complexes of cell surface molecules, referred to as human leukocyte antigen (HLA), or major histocompatibility complexes (MHCs), and peptides which are bound to said surface molecules. The peptides are derived from larger protein molecules which are processed by the cells which also present the HLA/MHC molecule. The HLA-associated peptides are short, encompassing 9-25 amino acids (Kropshofer & Vogt, Immunol. Today 18:77-82 (1997); Male et al., Advanced Immunology, chapters 6-10, J.P. Lipincott Company (1987)). They are indispensable for mounting an adaptive immune response as they activate the T-cells. The lack of T-cell recognition of peptides derived from tumour-specific antigens contributes to immune evasion and progressive growth of tumours (Boon et al., Ann. Rev. Immunol. 12, 337-265 (1994)).

With regard to their function, two classes of HLA-peptide complexes can be distinguished (Germain, Ann. N.Y. Acad. Sci. 754, 114-25 (1995)). HLA class I-peptide complexes can be expressed by almost all nucleated cells in order to attract CD8⁺-cytotoxic/cytolytic T cells (CTLs) which lyse cells that present the appropriate antigen, such as tumour cells or virus infected cells and thus play an essential role in eradicating said cells. HLA class II-peptide complexes are constitutively expressed only on so-called antigen presenting cells (APCs), such as B lymphocytes (short B-cells), macrophages or DCs. Of particular importance are DCs, which have the capacity to prime CD4⁺-T helper cells (Banchereau & Steinmann, Nature 392, 245-254 (1998)). CD4⁺-T helper cells secrete cytokines to stimulate macrophages and antigen producing B-cells, which present the appropriate antigen by HLA class II molecules on their surface. Moreover, DC can be licensed to activate optimally cytotoxic CD8⁺-T cells. This is accomplished through prior interaction of their HLA class II peptide complexes with CD4⁺-T helper cells (Ridge et al., Nature 393, 474-478 (1998)). CD4⁺ T cell help is therefore crucial for the induction and maintenance of strong CTL responses. Moreover, CD4⁺ T cells produce huge amounts of IF-γ which has been shown to inhibit angiogenesis by tumours. In addition, cytokines secreted by CD4⁺ T cells can recruit other immune cells with antitumour activity such as macrophages and neutrophils to the tumour site. Finally, CD4⁺ T cells can directly recognize and lyse HLA class II expressing tumour cells such as melanoma cells.

The apparent pivotal role of DCs in initiating immune responses has stimulated attempts to exploit DCs as vaccines, in particularly against cancer (Dallal & Lotze, Curr. Opin. Immunol. 12, 583-588 (2000); Orsini et al., Br. J. Haematol. 125, 720-728 (2004); Akiyama et al., Anticancer Res. 24, 571-577 (2004)). A key advance was the invention of techniques for differentiation of DCs in vitro from different sources including peripheral blood, e.g. adherent monocytes, or bone marrow-derived CD34⁺ stem cell precursors (EP 633929; EP 914415; EP 922758; EP 1412483; EP 1311658). DC differentiated and activated in vitro can be used for vaccination of cancer patients after co-culture with tumour cell-derived antigens or by employing analogous techniques. Pilot DC vaccination studies have successfully induced specific anticancer responses (Timmermann & Levy, Ann. Rev. Med. 50, 507-529 (1999); Yu et al., Cancer Res., 64, 4973-4979 (2004); Barbuto et al., Cancer Immunol. Immunother., Epub ahead of print, (Jun. 4, 2004); Avigan et al., Clin. Cancer Res. 10, 4699-4708 (2004); Banchereau et al., Cancer Res. 61, 6451-6458 (2001); Chang et al., Clin. Cancer Res. 8, 1021-1032 (2002); Fong et al., J. Immunol. 166, 4254-4259 (2001); Iwashita et al., Cancer Immunol. Immunother. 52, 155-161, Epub Feb. 6, 2003 (2003); Marten et al., Cancer Immunol. Immunother. 51, 637-644, Epub Oct. 3, 2002 (2002); Nestle et al., Nature Medicine 7, 761-765 (2001); Nestle et al., Nature Med. 4, 328-332 (1998)).

Vaccines based on the identification of tumour antigens include DCs primed with naked DNA, recombinant adeno- or vaccinia viruses, natural or recombinant proteins purified from the respective tumour cells or synthetic analogues of tumour peptides. The advantage of pulsing/loading DCs with antigenic tumour peptides rather than with genetic or protein precursors is that peptides can directly be loaded onto HLA molecules of DCs without further processing.

During the past decade, numerous peptides derived from tumour specific proteins and restricted by HLA class I molecules have been identified. In several clinical pilot vaccination studies, DCs from melanoma patients were pulsed with cocktails of melanoma peptides which, as yet, were exclusively HLA class I restricted (Nestle et al., Nature Med. 4, 328-332 (1998); Thurner, et al., J. Exp. Med. 190, 1669-1678 (1999)). However, there is increasing evidence that the efficacy and longevity of CTL responses against tumours can be increased by the recruitment of HLA class II-restricted T-helper cells.

Knowledge of HLA class II-restricted cancer antigens recognized by CD4⁺ T helper cells lags behind the identification of class I-restricted antigens (Wang, Trends in Immunol. 22, 269-276 (2001)). One reason is that transfection of cDNA libraries from tumour cells into target cells followed by usage of anti-tumour T-cells to identify the appropriate transfectants and antigenic epitopes—a method successfully employed with HLA class I molecules—is not effective because the encoded proteins do not travel to the HLA class II pathway in APCs.

An example for HLA class II presented human tumour-associated peptides are those derived from melanoma antigen (MAGE)-encoding genes, such as the MAGE-A3 peptides (WO 00/20581 and U.S. Pat. No. 6,716,809) presented by HLA-DR molecules or HLA-DP4. These peptides have already been used in clinical studies demonstrating that even in advanced melanoma patients strong CD4⁺ T-cell responses can be induced against melanoma cells. However, only 69% of melanomas express MAGE-A3 (Gaugler et al., J. Exp. Med. 179, 921-930 (1994)). It is also uncertain which role the MAGE-protein family, in particular MAGE-A3 plays during tumour development. An ideal target antigen (i.e. tumour associated protein) is widely expressed in melanomas (preferably in more than 90%), and its role during tumour development is defined: it plays a functional role in tumour development and formation of metastasis. The use of such target antigen reduces the risk of antigen loss considerably. MAGE-A3 is currently the best tumour antigen found in terms of clinical studies, i.e. it counts as gold standard for the development of improved tumour antigens. An ideal candidate for the development of melanoma-specific therapies however, would have a higher expression rate than MAGE-A3, i.e. it should be at least expressed in 90% of the tumours, in particular melanomas. Furthermore, an ideal antigen would be functionally important for the tumour cells as it has been shown for MCSP but not for MAGE-A3.

More recently HLA/MHC class II presented antigenic peptides derived from the translation factor eIF-4A, the IFN-γ-inducible protein p78, the cytoskeletal protein vimentin and the iron-binding surface protein melanotransferrin have been described (WO 2004/031230). By contacting DCs in vitro with necrotic melanoma cells under conditions stimulating antigen uptake, the DCs were antigen-loaded, and from the mature DCs the antigen-loaded MHC class II molecules were purified in order to isolate and identify the associated antigenic peptide. The peptides identified are also all found in other tissues than malignant growth. Vimentin, for example, a so-called intermediate filament protein, is most widely distributed, being present in the cytoplasm of many animal/human cells of mesodermal origin, including fibroblasts, endothelial cells, and white blood cells. In addition, many cells express it transiently during development. The translation factor eIF-4A is part of the translation machinery found in every single cell of an organism. The IFN-gamma-inducible protein p78, also called MxB, is produced by healthy cells in response to the virally induced presence of interferon. Melanotransferrin was one of the first surface marker proteins associated with human melanoma (Hellström et al., Int. J. Cancer 31, 553-555 (1983)). However it is also expressed in non-malignant cells, as an analysis of 50 tissues showed, and in particular it was found in the salivary glands, endothelial cells in the liver and brain and the sweat gland ducts (Richardson, Eur. J. Biochem. 267, 1290-1298 (2000)).

### SUMMARY OF THE INVENTION

In view of the above, it is desirable to obtain T-cell stimulatory, HLA class II-presented peptides from a protein which is strongly expressed in melanoma cells. Surprisingly it was found, that such peptides can be isolated from a specific amino acid region from MCSP, which is expressed in more than 90% of all melanomas. Since MCSP functions in adhesion, invasion and metastasis of melanoma are known, it represents an ideal candidate for the development of immunological anti-tumour/anti-melanoma therapies. The present invention provides peptides suitable for various aspects of cancer immunotherapy, including new vaccines and immunodiagnostic agents.

Furthermore, the present invention provides the use of MCSP, or of fragments, derivatives or variants thereof, as T-cell inducing agent. This agent can be used in cancer immunotherapy, especially as anti-cancer vaccine.

In more detail, the present invention provides:

(1) An antigenic T-cell stimulatory peptide (hereinafter “MCSP peptide”) which is derived from the melanoma-associated chondroitin sulfate proteoglycan (MCSP), has up to 100 amino acid residues, and comprises at least 8 amino acid residues out of the MCSP segment represented by amino acid residues 644 to 743 or 1270 to 1300 of MCSP of SEQ ID NO:1, or a functional variant or salt thereof;
(2) a fusion protein comprising as a first domain an MCSP peptide as defined in (1) above (MCSP domain) and at least one second domain;
(3) a nucleic acid sequence encoding anyone of the MCSP peptides and fusion proteins as defined in (1) and (2) above;
(4) a vector comprising the nucleic acid sequence as defined in (3) above;
(5) an isolated cell transfected or transformed with the vector as defined in (4) above and/or comprising the nucleic acid as defined in (3) above, whereby the cell is preferably selected from the group consisting of mammalian cells, preferably human or murine cells, more preferably primary cells such as melanoma cells, T-cells, antigen presenting cells including DCs, macrophages and B-cells, microorganism cells such as fungal cells, yeast cells and bacterial cells etc., insect cells and plant cells;
(6) use of anyone of the peptides and fusion proteins as defined in (1) and (2) above for research on and development of medicaments for cancer treatment, preferably for a treatment of melanoma and other MCSP expressing tumours such as breast cancer, notably lobular breast carcinoma, astrocytoma, glioma, glioblastoma, neuroblastoma, sarcoma and certain types of leukaemia;
(7) a method to generate stable mature dendritic cells (DCs) loaded with one or more of the peptides/proteins selected from (a) one or more of the MCSP peptides and functional variants as defined in (1) above, (b) one or more of the fusion proteins as defined in (2) above, and (c) the full length MCSP protein of SEQ ID NO:1 and fragments thereof, which method comprises the following steps:
(i) contacting isolated immature DCs or mature DCs with one or more of said peptides/proteins (a) to (c) defined above, to allow for uptake of said peptides/proteins, or
   contacting isolated immature DCs or mature DCs with one or more of the nucleic acid sequence encoding the peptides/proteins (a) to (c) defined above and/or with one or more vectors comprising nucleic acid sequences encoding the peptides/proteins (a) to (c) defined above, to allow for uptake and subsequent expression of the peptides/proteins in the DC; and
(ii) in case of immature DCs, maturing the DCs obtained in (i) by exposing them to a cytokine comprising maturation cocktail;
   (8) a method to generate a T-cell clone, preferably a CD4⁺ or CD8⁺ T cell clone, specific for one or more of the peptides/proteins selected from (a) one or more of the MCSP peptides and functional variants as defined in (1) above, (b) one or more of the fusion proteins as defined in (2) above, and (c) the full length MCSP protein of SEQ ID NO:1 and fragments thereof, which method comprises the following steps:
(i) contacting isolated T-cells, preferably isolated CD4⁺ or CD8⁺ T cells, with an antigen presenting cell (APC) presenting anyone of the peptides/proteins (a) to (c) as defined above, whereby the APC is selected from the group of B-lymphocytes, macrophages, and/or DCs, preferentially is a DC generated by the method of (7) above;
(ii) co-culturing the isolated T-cells with the APC for at least 30 days, whereby freshly prepared APCs are added for at least 3 times to the original co-culture, and the T-cells proliferate;
(iii) assessing the ability of the proliferating T-cells of (ii) to produce cytokines selected from the group of TNF-α, IFN-γ, GM-CSF, IL-2 in response to the addition of stimulator cells pulsed with anyone of the peptides/proteins (a) to (c) defined above, whereby the stimulator cells are selected from the group of autologous or allogenic immortalized B-cells, DCs, monocytes, and macrophages pulsed with anyone of the peptides/proteins as defined in (a) to (c) above;
(iv) cloning the TNF-α and/or IFN-γ producing T-cells of (iii) by limiting dilution culture in the presence of autologous or allogenic stimulator cells pulsed with anyone of the peptides/proteins (a) to (c) as defined above, and feeder cells, whereby the feeder cells are selected from the group of allogenic or autologous immortalized B-cells, LG2-EBV and allogenic or autologous PBMCS; and
(v) maintaining the isolated T-cell clone of step (iv) in the presence of feeder cells in culture medium comprising a maturation cocktail, preferably comprising interleukin-2 (IL-2), interleukin-7 (IL-7) and phytohemagglutinin (PHA);
   (9) a mature DC loaded with one or more of the peptides/proteins selected from (a) one or more of the MCSP peptides and functional variants as defined in (1) above, (b) one or more of the fusion proteins as defined in (2) above, and (c) the full length MCSP protein of SEQ ID NO:1 and fragments thereof, preferably the mature loaded DC is a DC obtainable by the method as defined in (7) above;
   (10) a T-cell clone, preferably a CD4⁺ or CD8⁺ T cell clone, specific for one or more of the peptides/proteins selected from (a) one or more of the MCSP peptides and functional variants as defined in (1) above, (b) one or more of the fusion proteins as defined in (2) above, and (c) the full length MCSP protein of SEQ ID NO:1 and fragments thereof, preferably said T-cell clone is a T cell clone obtainable by the method as defined in (8) above;
   (11) an antibody specific for anyone of the MCSP peptides, functional variants and fusion proteins as defined in (1) and (2) above;
   (12) a pharmaceutical or diagnostic composition comprising one or more of the MCSP peptides and fusion proteins as defined in (1) and (2) above, the nucleic acid sequences as defined in (3) above, the vectors as defined in (4) above, the transfected and/or transformed cells as defined in (5) above, the loaded mature DCs as defined in (9) above, the T-cell clones as defined in (10) above and/or the antibodies as defined in (11) above, and a pharmaceutically or diagnostically acceptable carrier;
   (13) use of one or more of the MCSP peptides and fusion proteins as defined in (1) and (2) above, the nucleic acid sequences as defined in (3) above, the vectors as defined in (4) above, the full length MCSP protein of SEQ ID NO:1 and fragments thereof, nucleic acids encoding the full length MCSP protein of SEQ ID NO:1 or fragments thereof, and vectors comprising nucleic acid sequences encoding the full length MCSP protein of SEQ ID NO:1 or fragments thereof
   (i) for the preparation of immune cells, preferably for the preparation of artificial APCs, mature loaded DCs, T-cell clones, B-cells secreting the antibodies as defined in (11) above and/or hybridomas secreting said antibodies; and/or
   (ii) as a diagnostic marker for cancer, preferably as a diagnostic marker for melanoma, including cutaneous and ocular melanoma, and other MCSP expressing tumours such as breast cancer, notably lobular breast carcinoma, astrocytoma, glioma, glioblastoma, neuroblastoma, sarcoma and certain types of leukaemia; and/or
   (iii) for the preparation of a medicament for preventing, treating and/or diagnosing cancer, preferably for preventing, treating and/or diagnosing melanoma, including cutaneous and ocular melanoma, and other MCSP expressing tumours such as breast cancer, notably lobular breast carcinoma, astrocytoma, glioma, glioblastoma, neuroblastoma, sarcoma and certain types of leukaemia;
   (iv) for the manufacturing of a medicament inducing a T cell response in a patient;
      (14) use of the transfected and/or transformed cells as defined in (5) above, the loaded mature DCs as defined in (9) above, the T-cell clones as defined in (10) above and/or the antibodies as defined in (11) above for the preparation of a medicament for preventing, treating and/or diagnosing cancer, preferably for preventing, treating and/or diagnosing melanoma, including cutaneous and ocular melanoma, and other MCSP expressing tumours such as breast cancer, notably lobular breast carcinoma, astrocytoma, glioma, glioblastoma, neuroblastoma, sarcoma and certain types of leukaemia;
      (15) use of one or more of the MCSP peptides and fusion proteins as defined in (1) and (2) above, the nucleic acid sequences as defined in (3) above, the vectors as defined in (4) above, for the manufacturing of a medicament stimulating the production of protective antibodies and/or immune cells;
      (16) a method for diagnosing and/or monitoring a disorder characterized by the expression of one or more of the MCSP peptides and fusion proteins as defined in (1) and (2) above, the full length MCSP protein of SEQ ID NO:1 and fragments thereof, comprising the following steps:
      (i) contacting a biological sample isolated from a subject having or suspected to have said disorder, with an agent that is specific for anyone of the MCSP peptides and fusion proteins as defined in (1) and (2) above, the full length MCSP protein of SEQ ID NO:1 and fragments thereof, preferably the agent being a T-cell clone as defined in (10) above or an antibody as defined in (11) above, and
      (ii) determining the interaction between the agent and the peptide;
      (17) a method for preventing or treating cancer, in particular melanoma, which method comprises administering to the patient an effective amount of one or more of the agents selected from the MCSP peptides and/or the fusion proteins as defined in (1) and (2) above, the nucleic acids as defined in (3) above, the vectors as defined in (4) above, the full length MCSP protein of SEQ ID NO:1 and fragments thereof, nucleic acid sequences encoding the full length MCSP protein of SEQ ID NO:1 or fragments thereof, and vectors comprising nucleic acid sequences encoding the full length MCSP protein of SEQ ID NO:1 or fragments thereof, the transfected/transformed cells as defined in (5) above, the loaded mature DCs as defined in (9) above, the T-cell clones as defined in (10) above, the antibodies as defined in (11) above, and/or the pharmaceutical composition as defined in (12) above; and
      (18) a method for preparing an MCSP peptide or a fusion protein as defined in (1) and (2) above, which method comprises culturing a cell as defined in (5) above and isolating the expressed peptide or fusion protein, or comprises chemical synthesis (i.e. solid phase) of the MCSP peptides or fusion proteins.

### BRIEF DESCRIPTION OF THE FIGURES

FIG. 1: Experimental protocol for activation of anti-MCSP CD4⁺ T-cells. Peripheral blood mononuclear cells (PBMCs) were isolated from a blood sample drawn with consent from a healthy donor. Using magnetic cell sorting (MACS) technology the CD4⁺ T-cells were isolated from the PBMCs, and seeded at 10⁵ cells per well of a microtiter plate. The CD4⁺ T-cells depleted fraction of the PBMCs was briefly cultured, in order to obtain adherent cells. Non-adherent cells were discarded, and the adherent cells were then exposed to a differentiation cocktail comprising GM-CSF and IL-4, and cultured for further 5 to 7 days, in order to obtain immature DCs, identifiable by their phenotype. The immature DCs were contacted for 1 h with 10 μg/ml of the candidate peptide derived from MCSP (SQVLFSVTRGAHYGEL (SEQ ID NO:12), VRYLSTDPQHHAYDTV (SEQ ID NO:13), GEALVNFTQAEVYAGN (SEQ ID NO:14)) or as a control peptide the MAGE-3.DP4 epitope, in order to obtain loaded DCs functioning as APCs. The ability of these APCs to induce in vitro activation and proliferation of specific CD4⁺ T-cells was tested by adding 10⁴ loaded DCs to each well of the microtiter plate already containing CD4⁺ T-cells. The mixed CD4⁺ T-cell/DCs were cultured on day 0 in the presence of IL-6, IL-12 and TNF-α and weekly restimulated with DCs freshly pulsed/loaded with the peptides and addition of IL-2 and IL-7. The CD4⁺ T-cell comprising microcultures were assessed on day 30 for their capacity to produce IFN-γ when stimulated with autologous target cells (Epstein Barr virus (EBV)-transformed B cells (EBV-B cells)) loaded with the relevant MCSP peptide or the control peptide, using an IFN-γ ELISA.

FIG. 2: Experimental protocol for activation of anti-MCSP CD4⁺ T-cells. Peripheral blood mononuclear cells (PBMCs) were isolated from a blood sample drawn with consent from a healthy donor. Using magnetic cell sorting (MACS) technology the CD4⁺ T-cells were isolated from the PBMCs, and seeded at 10⁵ cells per well of a microtiter plate. The CD4⁺ T-cells depleted fraction of the PBMCs was briefly cultured, in order to obtain adherent cells. Non-adherent cells were discarded, and the adherent cells were then exposed to a differentiation cocktail comprising GM-CSF and IL-4, and cultured for further 5 to 7 days, in order to obtain immature DCs, identifiable by their phenotype. The immature DCs were contacted overnight with 50 μg/ml of a 42 mer peptide derived from MCSP (MCSP-peptide₆₇₃₋₇₁₄) having the sequence LAQGSAMPILPANLSVETNAVGQDVSVLFRVTGALQFGELQK (SEQ ID NO:3).

The contact period was longer to allow for processing of the 42 mer and presentation of peptides derived there from. Furthermore, 6 h after exposure to the peptide, a cytokine maturation cocktail comprising IL-1β, IL-6, TNF-α and PGE₂ was added to the DCs, to induce maturation. The ability of these DCs to function as APCs capable of inducing in vitro activation and proliferation of specific CD4⁺ T-cells, was tested by adding 10⁴ loaded DCs to each well of the microtiter already containing CD4⁺ T-cells. The mixed CD4⁺ T-cell/DCs were cultured on day 0 in the presence of IL-6, IL-12 and TNF-α and weekly restimulated with DCs freshly pulsed/loaded with the peptide and addition of IL-2 and IL-7. The CD4⁺ T-cell comprising microcultures were assessed on day 30 for their capacity to produce IFN-γ when stimulated with autologous target cells (Epstein Barr virus (EBV)-transformed B cells (EBV-B cells)) loaded with the MCSP peptide or an irrelevant control peptide, using an IFN-γ ELISA. Three peptide-specific CD4⁺ T-cell-clones were obtained, and the following experiments were conducted with clone C2/25 (clone 25).

FIG. 3: Autologous EBV-B-cells of donor 4800 were pulsed/loaded overnight with the 42 mer MCSP-peptide₆₇₃₋₇₁₄ (5 μM) or a control peptide, washed and used as stimulator cells. 4×10³ CD4⁺ T-cells were co-incubated with 1.5×10⁴ stimulator cells for 20 h, before the IFN-γ concentration in the supernatant was measured by an ELISA. Values represent means of triplicates.

FIG. 4: Autologous EBV-B-cells (1.5×10⁴) were pulsed/loaded with overlapping peptide fragments of the 42 mer MCSP-peptide₆₇₃₋₇₁₄ (1 μM) for 1 h, washed and co-cultured for 20 h with the CD4⁺ T-cell-clone 25 in order to test for recognition and IFN-γ secretion. An IFN-γ ELISA was performed.

FIG. 5: Fine-specificity of the shortest MCSP epitope recognized by CD4⁺ T-cell-clone 25. Autologous EBV-B cells (1.5×10⁴) were pulsed for 1 h with a panel of truncated peptides (1 μM) and tested for recognition by the CD4⁺ T cell clone. IFN-γ production was measured after overnight co-culture by ELISA. The set of truncated peptides was derived from the overlapping peptide VGQDVSVLFRVTGALQ (SEQ ID NO:9). The peptides were truncated for up to 7 amino acids at the N- or C-terminus. Truncations for up to 3 amino acids at both N- and C-terminus were tolerated. Truncation going beyond the D at amino acid position 696 (SEQ ID NO:1) or the A at amino acid position 706, at the N terminus or C-terminus, respectively, resulted in the loss of recognition by the T cell clone, as measured by an IFN-γ ELISA.

FIG. 6: Autologous EBV-B cells (1.5×10⁴) pulsed with 1 μM of the 16-mer MCSP peptide VGQDVSVLFRVTGALQ (SEQ ID NO:9) were used as stimulator cells in the presence of different blocking antibodies (anti-DR, anti-DQ or anti-DP). Also a control with no antibody (no Ab) was set up. All antibodies were used at a final concentration of 5 μg/ml each. IFN-γ production of the CD4⁺ T cells was measured after overnight co-culture (20 h) by ELISA.

FIG. 7: 1.5×10⁴ cells of several EBV-B cell lines (LP2-, LB1981-, R12-, PV6-, AC 42- and 4800-EBV) with different HLA class II molecules (HLA-DR11 positive or HLA-DR11 negative) were pulsed/loaded for 1 h with MCSP peptide VGQDVSVLFRVTGALQ (SEQ ID NO:9) (1 μM, white bar) and tested for recognition by the CD4⁺ T cell clone. As control, the cells were pulsed in the absence of protein (black spotted bar). IFN-γ production of the CD4⁺ T cells was measured after overnight co-culture (20 h) by ELISA.

FIG. 8: EBV-B cell lines HLA-DR11 positive (4800- and MVGS EBV) or not (MMDH EBV) were transduced with a retrovirus coding for an Ii.-MCSP fusion protein (spotted bars) or an Ii.-MAGE-3 protein (white bars) as a control The fusion protein comprised amino acid residues 392 to 748 of the MCSP. Clone 25 was then co-cultured with 1.5×10⁴ stimulator cells for 20 h. IFN-γ production of the CD4⁺ T cells was measured after overnight co-culture by ELISA.

FIG. 9: Clone 25 was stimulated by HLA-matched or -mismatched MCSP-expressing melanoma cell lines (2×10⁴). IFN-γ production of the CD4⁺ T cells was measured after overnight co-culture by ELISA.

FIG. 10: CD4⁺ T cells (100,000 per 96 round-bottomed microwell) of donor 11325 were stimulated with autologous monocyte-derived dendritic cells (10,000 per well) loaded overnight with the long TAT-MCSP-peptide (SEQ ID NO:54; 10 μM). After 3 weekly re-stimulations, microcultures were tested for their IFN-γ production when stimulated with autologous EBV-B cells loaded with the MCSP peptide or a control peptide.

FIG. 11: Autologous EBV-B cells of donor 11325 were loaded overnight with the long TAT-MCSP-peptide (SEQ ID NO:54; 10 μM) or a control peptide, washed and used as stimulator cells. 4,000 CD4⁺ T-cells were co-incubated with 15,000 stimulator cells and after 20 h the IFN-γ concentration in the supernatant was measured by ELISA.

FIG. 12: Autologous EBV-B cells (15,000) were pulsed with overlapping truncated peptides (5 μg/ml) for 1 h or in the case of the long TAT-MCSP-peptide (SEQ ID NO:54) overnight at 10 μM, washed and tested for recognition by the CD4⁺ T-cell clone (4,000 cells per well) after 20 h co-culture by IFN-γ-ELISA. Values shown are the mean of duplicate determinations, bars, SD.

FIG. 13: Autologous EBV-B cells (15,000) were pulsed for 1 h with a panel of truncated peptides (5 μg/ml) and tested for recognition by the CD4⁺ T cell clone (4,000 cells per well). IFN-γ production was measured after overnight co-culture by ELISA. Values shown are the mean of duplicate determinations, bars, SD.

FIG. 14: Autologous EBV-B cells (15,000) pulsed with 16-mer MCSP peptide (aa 1281-1296 (SEQ ID NO:48), 5 μg/ml) were used as stimulator cells in the presence of different blocking antibodies. All antibodies were used at a final concentration of 5 μg/ml each. IFN-γ production by CD4⁺ T cells (4,000 cells per well) was measured after overnight co-culture by ELISA.

FIG. 15: Several EBV-B cell lines with different HLA class II molecules were pulsed with peptide MCSP₁₂₈₁₋₁₂₉₆ (SEQ ID NO:48; 5 μg/ml) and tested for recognition (at 15,000 cells per well) by the CD4⁺ T cell clone (4,000 cells per well). IFN-γ production by CD4⁺ T cells was measured after overnight co-culture by ELISA.

FIG. 16: Clone 3 was stimulated by HLA-matched (ER-MEL-4) or -mismatched MCSP-expressing melanoma cell lines (20,000) which had been thawed 48 h before to allow the formation of a confluent cell layer. IFN-γ production by CD4⁺ T cells (4,000 cells per well) was measured after overnight co-culture by ELISA.

FIG. 17: CD8⁺ T cells (150,000 per 96 round-bottomed microwells) of donor 11325 were stimulated with autologous monocyte-derived dendritic cells (15,000 per well) loaded overnight with the long TAT-MCSP-peptide (SEQ ID NO:54; 10 μM). After 3 weekly restimulations microcultures were tested for their IFN-γ production when stimulated with autologous EBV-B cells loaded with the TAT-MCSP peptide (SEQ ID NO:54) or a control peptide.

FIG. 18: Autologous EBV-B-cells of donor 11325 were loaded overnight with the long TAT-MCSP-peptide (SEQ ID NO:54; 10 μM) or a control peptide, washed and used as stimulator cells. Aliquots of approximately 4,000 CD8⁺ T-cells from each 96-well microculture were co-incubated with 15,000 stimulator cells and after 20 h the IFN-γ concentration in the supernatant was measured by ELISA. Values shown represent duplicate determinations from two representative positive tested microcultures out of 96 tested. Bars represent standard deviations.

### DETAILED DESCRIPTION OF THE INVENTION

The aim of the present invention was the identification of T-cell epitopes from the melanoma-associated chondroitin sulfate proteoglycan (MCSP), a tumour antigen with potential benefit for vaccination and immunomonitoring of cancer, especially of melanoma patients. The identification of tumour antigens recognized by cytolytic CD8⁺ T cells (CTLs) on human tumour cells has opened new avenues in cancer immunotherapy. There is consensus that the induction of both tumour-specific CTLs and CD4⁺ T helper cells is necessary for an optimal antitumour immunity. Unfortunately, only a few tumour-specific helper T cell epitopes have been described so far. Therefore the present invention focuses on the identification of melanoma antigens recognized by CD4⁺ T cells. One interesting candidate antigen is the human melanoma-associated chondroitin sulfate proteoglycan (MCSP), which is expressed on >90% of human melanoma tissues and induces strong humoral responses in mice. The demonstration of humoral anti-MCSP immunity in a mouse and a human model implicated the co-existence of MCSP-specific CD4⁺ T-cells, therefore the present invention is focused on the identification and verification of MCSP-specific CD4⁺ T-cell responses. In addition, the presence of MCSP-specific CD8⁺ T-cell responses is demonstrated.

### DEFINITIONS

A “T cell” in the context of present invention is a CD3+ lymphocyte. Preferably, the T cells of present invention are CD4⁺ or CD8⁺ cells, more preferably CD4⁺ cells.

The expression “antigenic T-cell stimulatory peptide” refers to the ability of a peptide to be recognized by a T-cell when bound to a given HLA molecule and to stimulate the T-cell to secrete cytokines and/or to proliferate and/or to display lytic activity. The expression “functional variant” refers to the antigenic T-cell stimulatory peptide, comprising at least one amino acid addition or substitution not affecting the ability of the peptide to stimulate a T-cell. Preferably an addition arises from adding between 1 to 15 amino acids, preferably 1 to 10, most preferably 5 to 10 amino acids anywhere to the 100 amino acid core peptide comprising sequence at amino acid position 644 to 743 (SEQ ID NO:1), or from adding between 1 to 15, preferably 1 to 10, most preferably 5 to 10 amino acids to the N- and/or C-terminus of the peptide fragments derived from the amino acid region 673-714 of SEQ ID NO:1. The term “substitution” refers to the replacement of an amino acid with an homologous amino acid, which are known to the person skilled in the art. A homologous substitution is also referred to as “conservative amino acid change”. Furthermore, it also refers to the replacement of an amino acid with the corresponding D-amino acid, or a derivatized amino acid. A “derivatized amino acid” is an amino acid which comprises a modified functional group, such as a free amino group which has been chemically modified to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazol nitrogen of histidine may be derivatized to form N-imbenzylhistidine. Also included are naturally-occurring amino acid derivatives of the twenty standard amino acids. For example: 4-hydroxyproline may be substituted for proline, 5-hydroxylysine may be substituted for lysine, homoserine may be substituted for serine, 3-methylhistidine may be substituted for histidine, and ornithine or citrulline may be substituted for lysine. The functional variant may comprise up to 15 substitutions, preferably 1 to 10, most preferably 1 to 5 substitutions.

“Fusion peptide” according to the invention refers to peptides where a first domain comprising an antigenic T cell stimulating peptide (i.e. the MCSP domain) is directly or through a linker peptide fused to a second function protein or peptide donor.

The term “one or more of the MCSP peptides and fusion proteins” includes the single MCSP peptide or fusion protein and also mixtures out of said MCSP peptides and/or fusion proteins of the invention. Where applicable, the term “one or more” is to be construed similarly for the nucleic acids and vectors, for the cells and for the antibodies of the invention.

The expression “endosomal targeting signal” refers to a short peptide sequence, in general comprising up to 30 amino acids, which directs the protein and/or peptide with which its functionally linked into the endosome. “Functionally linked” is here to be understood to refer to a peptide bond between the sequence comprising the endosomal targeting signal sequence and the peptide according to the invention. A short peptide linker may lie between the endosomal targeting sequence and the peptide/protein sequence to be targeted into the endosome.

The expression “nucleic acid” refers to any nucleic acid known to a person skilled in the art, including DNA and RNA, whereby the nucleic acid may be double-stranded, single-stranded, circular and/or linear. It also includes nucleic acid molecules with modifications to their bases as well as the sugar-phosphate backbone.

The term “vector” is generally understood by a person skilled in the art and comprises a DNA molecule, or its corresponding RNA molecule, derived from a plasmid, a bacterial phage, or a mammalian or insect virus, into which fragments of DNA may be inserted or cloned. A vector comprises one or more unique restriction enzyme sites and may be capable of autonomous replication. Furthermore the vector is capable of expressing the inserted or cloned fragment by providing transcription control elements, such as a promoter and transcription termination signals. The DNA fragment inserted in to the vector, here the DNA coding for the peptide according to the invention, is functionally linked to said transcription control elements. In addition the vector may also comprise a nucleic acid sequence encoding HLA-DR11. Such a vector, when transfected into a cell, gives rise to an artificial antigen presenting cell. Examples of suitable vectors include retroviral vectors, vaccinia vectors, adenoviral vectors, herpes virus vectors, fowl pox virus vectors, plasmids, baculovirus transfer vectors.

The terms “transfected” and “transformed” refer to the introduction of a nucleic acid into a cell. Various transfection and transformation methods are known to the person skilled in the art. The term “transfection” generally describes the introduction of a nucleic acid into a mammalian cell, whereas the term “transformation” describes the uptake of a nucleic acid by a microbial cell such as fungal cells or prokaryotes. The means by which the nucleic acids are introduced into the cell include microinjection, lipofection, electroporation, calcium phosphate transfection, DEAE-dextran transfection or infection with a recombinant virus harbouring said nucleic acid (Sambrook et al. in “Molecular Cloning. A Laboratory Manual”, Cold Spring Harbor Press, Plainview, N.Y. (1989)).

A CD4⁺ T-cell clone is said to be “specific” for a peptide, if upon exposure to the peptide bound to a HLA-class II molecule, the CD4⁺ T-cell recognizes the HLA-peptide-complex with its T cell receptor resulting in cytokine secretion by the CD4⁺ T-cell, in particular TNF-α and/or IFN-γ secretion, and/or other cytokines, as well as proliferation of the CD4⁺ T-cell.

Analogously, a CD8⁺ T cell clone is said to be “specific” for a peptide, if upon exposure to the peptide bound to a HLA-class II molecule, the CD8⁺ T-cell recognizes the HLA-peptide-complex with its T cell receptor resulting in cytokine secretion by the CD8⁺ T-cell, in particular TNF-α and/or IFN-γ secretion, and/or other cytokines, as well as proliferation of the CD8⁺ T-cell.

The term “antigen presenting cell” (APC) is generally understood by the person skilled in the art. It refers to highly specialized cells that can process antigens and display their peptide fragments on the cell surface together with molecules required for lymphocyte activation. The main APCs for T cells are DCs, macrophages, and B-cells, while the main APCs for B-cells are follicular DCs. In the sense of the present invention the term APC also comprises artificial APCs, which can be generated by co-expressing the nucleic acids encoding the molecules required for lymphocyte activation, in particular HLA-class II molecules, such as HLA-DR11, as well as the nucleic acids encoding the peptides according to the invention, in cells not normally functioning as APCs. The co-expression may be achieved via transfection with a single vector comprising the coding sequences for both, the peptide as well as the HLA-class II molecule, e.g. HLA-DR11; or by co-transfection of two individual vector molecules, one encoding anyone of the peptides according to the invention and the other one the HLA-class II molecule, e.g. HLA-DR11.

The expression “stimulator cell” refers to cells used in assays to test the ability of T-cells to respond to the antigenic peptide according to the invention. In general stimulator cells are characterized by the expression of antigenic peptides bound to the groove of HLA class I/II molecule and the expression of costimulatory molecules such as CD80 and CD86. Stimulator cells are generally selected from the group of Epstein-Barr virus (EBV) transformed autologous or allogenic B cells (in short: EBV-B cells) pulsed with the peptide according to the invention. Autologous cells are preferred. Due to the transformation with EBV the B cells are immortalized. Stimulator cells can also be selected from the group of macrophages, PBMCS, DCs, and CD40-ligand stimulated B-cells. The expression “pulsed”, “pulsed with”, “loaded”, “loaded with”, “pulsed/loaded” or “pulsed/loaded with” refers to cells displaying anyone of the peptides according to the invention. The pulsing/loading is achieved by exposing cells, e.g. DCs or EBV-B cells, to said peptide for an amount of time sufficient to allow uptake. In general, 1 h is sufficient to achieve uptaking and displaying, but the exposure time may be expanded for up to 20 h.

In the context of the present invention the expression “assessing the ability of proliferating T-cells”—notably the CD4⁺ and CD8⁺ T cells—“to produce TNF-α and/or IFN-γ” is meant to refer to various assays allowing measurement of said cytokines. To the person skilled in the art it is clear that other cytokines, for example GM-CSF or IL-2, can be assessed in analogous ways. In particular, the “assessing” can be achieved through a cytokine-specific ELISA assay, but also through other methods, such as bioassays, in which cells responsive to the secreted cytokine are tested for responsiveness (e.g. proliferation) in the presence of a test sample or the ELISPOT assay, cytokine bead arrays, quantitative real-time PCR for cytokines or intracytoplasmatic cytokine staining.

The term “feeder cells” refers to cells which may secrete protein factors or give other types of stimulatory signals supporting the proliferation of T cells. Feeder cells may be selected from the group of immortalized B-cells, such as allogenic as well as autologous B-cells, PBMCs and CD40-ligand activated B-cells. Preferably the feeder cells are LG2-EBV cells.

The term “cloning . . . the T-cell” refers to obtaining a T-cell population derived from a single T-cell, whereby all cells of the population have an identical genotype and phenotype. Cloning is achieved by limiting dilution culture, i.e. harvesting a T-cell population determined to secrete TNF-α, IFN-γ and/or other cytokines, e.g. GM-CSF and diluting the harvested T-cell population by a factor 10¹ to 10⁸, plating out and co-culturing said diluted T-cell populations in the presence of feeder cells. Individual T-cell clones can be obtained in this way.

The term “antibody” is generally understood by the person skilled in the art. In particular it refers to proteins that bind specifically to particular antigens, in the context of the present invention to those which bind to the peptides according to the invention, and are produced in response to immunization with the antigen. They bind to and neutralize cells displaying the antigen and prepare them for uptake and destruction by phagocytes. The term “protective antibody” refers to an antibody which protects an organism from harmful matter, including tumour cells expressing and displaying an antigenic peptide according to the invention. As used herein, the term “antibody” refers to polyclonal antibodies, monoclonal antibodies, humanized antibodies, single-chain antibodies, and fragments thereof such as Fab, F(ab′)2, Fv, and other fragments which retain the antigen binding function and specificity of the parent antibody. The term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)2, Fv, and others which retain the antigen binding function and specificity of the antibody.

As used herein, the term “human antibodies” means that the framework regions of an immunoglobulin are derived from human immunoglobulin sequences. As used herein, the term “single chain antibody fragments” (scFv) refer to antibodies prepared by determining the binding domains (both heavy and light chains) of a binding antibody, and supplying a linking moiety which permits preservation of the binding function. This form, in essence, is a radically abbreviated antibody, having only that part of the variable domain necessary for binding to the antigen. Determination and construction of single chain antibodies are described in U.S. Pat. No. 4,946,778 by Ladner et al.

The expression “pharmaceutically acceptable carrier” refers to all known substances used for the formulation of a medicament, not themselves being an active ingredient of the medicament. The expression “diagnostically acceptable carrier refers to substances used for the formulation of diagnostika which are not interfering with the reaction indicative for the diagnostically targeted disease.

The term “vaccine” refers to a composition either used prophylactically or therapeutically to prevent or treat diseases associated with the expression of the peptides according to the invention or with the expression of MCSP itself. In particular the vaccine of present invention is used against melanoma and other MCSP expressing tumours such as breast cancer, notably lobular breast carcinoma, astrocytoma, glioma, glioblastoma, neuroblastoma, sarcoma and certain types of leukaemia. The vaccine is characterized in that it triggers an immune response, in particular a cellular immune response. As adjuvant the vaccine may comprise Freund's complete adjuvants, Freund's incomplete adjuvants, Montanide ISA Adjuvants (Seppic, Paris, France), Ribi's Adjuvants (Ribi ImmunoChem Research, Inc., Hamilton, Mont.), Hunter's TiterMax (CytRx Corp., Norcross, Ga.), Aluminum Salt Adjuvants, Gerbu Adjuvant (Gerbu Biotechnik GmbH, Gaiberg, Germany/C-C Biotech, Poway, Calif.), MPL (Glaxo Smithkline), AS02B (Glaxo Smithkline), QS21 (Glaxo Smithkline) and/or Toll like receptor agonists such as imiquimod (3M Medica, Neuss, Germany).

The expression “immune cell” refers to any cell participating in the immune response. In particular it refers to B-cells, T-cells, monocytes, macrophages, dendritic cells, NK-cells and/or NKT-cells.

The term “melanoma” refers to all kinds of melanoma including cutaneous melanoma, ocular melanoma, metastatic melanoma, melanomas derived from either melanocytes or melanocyte related nevus cells, melanocarcinoma, melanoepitheliomas, melanosarcomas, melanoma in situ, superficial spreading melanoma, nodular melanoma, lentigo maligna melanoma, acral lentiginous melanoma, invasive melanoma or familial atypical mole and melanoma (FAM-M) syndrome. Such melanomas in mammals may be caused by chromosomal abnormalities, degenerative growth and developmental disorders, mitogenic agents, ultraviolet radiation (UV), viral infections, inappropriate tissue expression of a gene, alterations in expression of a gene, and presentation on a cell, or carcinogenic agents.

The term “certain kinds of leukaemia” refers to adult acute lymphoblastic leukaemia (ALL) and childhood acute myeloid leukaemia (AML).

The expression “diagnostic marker for cancer” refers to molecules specifically found on cancerous growth. These may be proteins and peptides newly expressed, mutated, or aberrantly expressed in tumour cells. They allow to distinguish the tumour cell from a healthy cell, which does not express said proteins or peptides.

The term “medicament” in the context of the present invention refers to a vaccine, a diagnostic agent as well as to any other therapeutically active pharmaceutical composition.

The term “immunomonitoring” refers to a diagnostic monitoring procedure, whereby cells of the immune system capable of binding to the peptide of the invention, such as B-cells or T-cells are quantified. High numbers of these cells specific for anyone of the peptides according to the invention are likely to be diagnostic of a relevant disease, such as a tumour, in particular melanoma, or an indication that these cells are involved in immunity to the disease. In particular useful for immunomonitoring may be multimers (dimers, trimers, tetramers, pentamers, hexamers or oligomers) of a class II HLA molecule comprising a covalently or non-covalently bound peptide according to the invention, which is conjugated with a detectable label. A label may be selected from the group of fluorescent moieties, radionuclides, or enzymes that catalyze a reaction resulting in a product that absorbs or emits light of a defined wavelength. Such a multimer may be used to quantify in vitro T cells or B-cells from a subject, e.g. a human patient, bearing cell surface receptors that are specific for, and therefore will bind such multimers.

The expression “biological sample” refers to any material isolated from a subject, e.g. a human patient. In particular it refers to biopsy material and/or a blood sample obtained from a subject.

The term “fragment of the full length MCSP protein” in the context of present invention designates a fragment of the full length amino acid sequence of MCSP as represented by SEQ ID NO:1 which is immunogenic, i.e. which is able to stimulate an immune response in mammals, preferably in humans. Said fragment contains a chain of consecutive amino acids of said SEQ ID NO:1, preferably at least 10 consecutive amino acids, more preferably at least 13 consecutive amino acids. Its maximum length is 100 amino acids, more preferably 42 amino acids.

The present invention discloses an antigenic T-cell stimulatory peptide from the melanoma-associated chondroitin sulfate proteoglycan (MCSP) (SEQ ID NO:1) of 100 amino acids in length comprising amino acids 644 to 743 of MCSP (SEQ ID NO:1), and/or a fragment thereof of at least 8 amino acids, and/or a functional variant thereof comprising one or more amino acid additions or substitutions. It has to be noted that the sequence given as SEQ ID NO:1 corresponds to the native protein, i.e. comprises the 29 amino acid signal peptide of the MCSP. The nucleic acid encoding the MCSP as given in SEQ ID NO:1 is shown in SEQ ID NO:2.

The MCSP peptide of embodiment (1) comprises at least 10, preferably at least 12, more preferably at least 13 amino acid residues. In one preferred aspect, the peptide of embodiment (1) comprises the MCSP fragment represented by amino acid residues 695 to 705 of SEQ ID NO:1 (QDVSVLFRVTG) or by amino acid residues 1285 to 1295 of SEQ ID NO:1 (GYLVMVSRGAL). In an even more preferred aspect, it comprises

(i) at least 13 consecutive amino acid residues out of the MCSP segment represented by amino acid residues 644 to 743 of SEQ ID NO:1, preferably comprises the amino acid residues 695 to 705 of SEQ ID NO:1; or
(ii) at least 12 consecutive amino acid residues out of the MCSP segment represented by amino acid residues 1270 to 1300 of SEQ ID NO:1, preferably comprises the amino acid residues 1285 to 1295 of SEQ ID NO:1.

In particular the present invention relates to a peptide fragment of 13 to 42 amino acids in length, which is derived from the 100 amino acid region at amino acid position 644 to 743 of MCSP (SEQ ID NO:1). The peptide of embodiment (1) is preferably derived from the MCSP fragment represented by amino acid residues 673 to 714 or 1270 to 1300 of SEQ ID NO: 1. Preferred peptide fragments comprise a sequence selected from the following amino acid sequences:

(SEQ ID NO:3)

LAQGSAMPILPANLSVETNAVGQDVSVLFRVTGALQFGELQK,

(SEQ ID NO:4)

LAQGSAMPILPANLSV;

(SEQ ID NO:5)

SAMPILPANLSVETNA;

(SEQ ID NO:6)

ILPANLSVETNAVGQD;

(SEQ ID NO:7)

NLSVETNAVGQDVSVL;

(SEQ ID NO:8)

ETNAVGQDVSVLFRVT;

(SEQ ID NO:9)

VGQDVSVLFRVTGALQ;

(SEQ ID NO:10)

VSVLFRVTGALQFGEL;
or

(SEQ ID NO:11)

FRVTGALQFGELQK.
Especially preferred peptides have
a sequence selected from

(SEQ ID NO: 3)

LAQGSAMPILPANLSVETNAVGQDVSVLFRVTGALQFGELQK,

(SEQ ID NO:8)

ETNAVGQDVSVLFRVT
and

(SEQ ID NO:9)

VGQDVSVLFRVTGALQ,

Said peptide fragments may be shortened by up to three C-terminal and/or N-terminal amino acids, without loosing their antigenic T-cell stimulatory function. Preferably, said peptide fragments are not shortened on their termini.

Moreover in particular the present invention relates to the peptide fragment of 12 to 30 amino acids in length, derived from the 31 amino acid region at amino acid position 1270 to 1300 of MCSP (SEQ ID NO:1). Preferred peptide fragments comprise a sequence selected from the following amino acid sequences:

(SEQ ID NO:36)

PPADIVFSVKSPPSAGYLVMVSRGALADEPP,

(SEQ ID NO:37)

PPSAGYLVMVS,

(SEQ ID NO:38)

PPSAGYLVMVSR,

(SEQ ID NO:39)

PPSAGYLVMVSRG,

(SEQ ID NO:40)

PPSAGYLVMVSRGA,

(SEQ ID NO:41)

PPSAGYLVMVSRGAL,

(SEQ ID NO:42)

PPSAGYLVMVSRGALA,

(SEQ ID NO:43)

PSAGYLVMVSRGALA,

(SEQ ID NO:44)

SAGYLVMVSRGALA,

(SEQ ID NO:45)

AGYLVMVSRGALA,

(SEQ ID NO:46)

GYLVMVSRGALA,

(SEQ ID NO:47)

YLVMVSRGALA,

(SEQ ID NO:48)

PPADIVFSVKSPPSAG,

(SEQ ID NO:49)

IVFSVKSPPSAGYLV,

(SEQ ID NO:50)

SVKSPPSAGYLVMVSR,

(SEQ ID NO:51)

GYLVMVSRGALADEPP.

Especially preferred peptides have a sequence selected from

(SEQ ID NO:36)

PPADIVFSVKSPPSAGYLVMVSRGALADEPP,

(SEQ ID NO:41)

PPSAGYLVMVSRGAL,

(SEQ ID NO:42)

PPSAGYLVMVSRGALA,

(SEQ ID NO:43)

PSAGYLVMVSRGALA,

(SEQ ID NO:44)

SAGYLVMVSRGALA,

(SEQ ID NO:45)

AGYLVMVSRGALA,

(SEQ ID NO:46)

GYLVMVSRGALA,
and

(SEQ ID NO:51)

GYLVMVSRGALADEPP.

Said peptide fragments may be shortened by up to three C-terminal and/or N-terminal amino acids. Preferably, said peptide fragments are not shortened on their termini.

Anyone of the above described peptides and/or peptide fragments and/or functional variant thereof, can be found either in full-length or in fragmented form associated with HLA-DR molecules, e.g. with HLA-DR-11, i.e. anyone of these peptides can be presented by HLA-DR11. Said peptides may also be found associated with HLA-DP molecules.

In a further embodiment the above described peptides comprise at least one conservative amino acid exchange, at least one amino acid replaced by a D-amino acid, and/or at least one amide bond selected from the group of psi[CH₂NH]-reduced amide peptide bonds, psi[COCH₂]-ketomethylene peptide bond, psi[C(CN)NH]-(cyanomethylene) amino peptide bond, a psi[CH₂CH(OH)]-hydroxyethylene peptide bond, a psi[CH₂O)]-peptide bond and psi[CH₂S]-thiomethylene peptide bond. Peptides comprising any such modification are more stable in comparison to unmodified peptides. In particular they have an extended plasma half life. This is of particular advantage because it allows for administration of lower dosages of a medicament comprising said peptides.

Anyone of the above described MCSP peptides may be part of the fusion protein according to embodiment (2), i.e. may form the MCSP domain of said fusion protein. Said fusion protein preferably comprises one second domain, but it is to be understood that it may also comprise more than one second domains which may be same or different and selected, e.g., from those domains mentioned below.

The at least one second domain of the fusion protein, i.e. the fusion partner(s) of the MCSP domain, preferably is a protein and/or peptide and comprises an endosomal targeting signal, and/or is selected from the group of the human invariable chain (Ii), a peptide fragment thereof comprising amino acid residues 1-80, the lysosome-associated membrane protein (LAMP-1) and DC-LAMP. Thus the fusion protein comprises at least two domains, viz. the MCSP domain comprising anyone of the MCSP peptides according to the invention, and the second domain comprising a protein or peptide with an endosomal targeting signal. The order of the domains will be chosen to allow for functionality of the domains, i.e; the endosomal targeting signal must be capable of directing the fusion protein into the endosome.

The first and the at least one second domain of the fusion protein may be connected directly (i.e. by a peptide bond) or through a linker peptide. Suitable linker peptides have a length of 1 to 20, preferably of 1 to 12 amino acid residues and are preferably comprised of flexible hydrophobic amino acid residues. Particularly useful linkers are polypeptides such as poly-Gly, poly-Gly-Ser and the like.

In a further preferred embodiment the at least one second domain of the fusion protein (2) is a protein transduction domain, including protein transduction domains of the HIV TAT protein or variants thereof. Preferred protein transduction domains within the present invention are variants of the HIV TAT protein, particularly preferred is the HIV TAT protein variant YARAAARQARA (SEQ ID NO:53) linked to the N-terminus of the MCSP domain.

A particularly preferred fusion protein is a 42-mer having the sequence YARAAARQARAPPADIVFSVKSPPSAGYLVMVSRGALADEPP (SEQ ID NO:54).

In a further embodiment the present invention provides a nucleic acid sequence encoding anyone of the MCSP peptides and/or peptide fragments and/or functional variants and/or fusion proteins described hereinbefore. A vector comprising any such nucleic acid sequence is also part of the invention. An alternative embodiment of said vector is a vector also comprising a nucleic acid sequence encoding HLA-DR11. Such a vector enables the generation of artificial APCs, by transfecting a cell normally not expressing the HLA-DR11 complex with said vector. If a fibroblast is transfected with said vector, the fibroblast will be turned into an artificial APC. The cell, e.g. the fibroblast, transformed with said vector will surface present the recombinant HLA-DR11 molecule as well as the peptide encoded by said vector. The peptide will also occur bound to the recombinant HLA-DR11 molecule.

A further embodiment of the invention is an isolated cell transfected or transformed with anyone of the vector molecules described above, and/or a cell comprising the nucleic acid molecule according to the invention. Said cell is preferably selected from the group of insect cells, plant cells, mammalian cells, preferably a human cell or murine cell, most preferably primary cells such as fibroblast, melanoma cells, DCs, B cells, macrophages, and microorganism cells such as eukaryotic cells including fungal cells (e.g. yeast cells, etc.) and prokaryotic cells, such as E. coli.

Also an preferred aspect of embodiment (7) of the present invention is a method to generate stable mature dendritic cells (DCs) loaded with anyone of the MCSP peptides and fusion proteins as described above, the full length MCSP protein of SEQ ID NO:1 and fragments thereof. Preferably, said cells are loaded with one or more of the MCSP peptides and fusion proteins as described above and the full length MCSP protein, more preferably with the peptides or fusion proteins as described above. The preferred method comprises the following steps: (i) contacting isolated immature DCs with anyone of the peptides or fusion proteins or the full length protein (at concentrations of 0.01 to 1000 μM) described above to allow for uptake of said peptides or fusion proteins or full length protein, or contacting them with anyone of the nucleic acid molecules and/or the vector molecule described above, to allow for uptake and subsequent expression of the peptide in the DC; and (ii) maturing the contacted DCs by exposing them to a cytokine comprising maturation cocktail (IL-1β, IL-6, PGE₂ and TNF-α) and/or monocyte conditioned medium. The contacting phase may vary in time, but the immature DCs are brought in contact with anyone of the peptides or fusion proteins according to the invention for at least 1 h, however, the contact phase may be extended for up to 30 h, whereby 20 h are preferred. The cytokine comprising maturation cocktail is essentially composed of IL-1β, IL-6, TNF-α, and PGE₂, and is added only if a longer contact phase was chosen. Then it is added at about 6 h past the initial contacting and left on the cells until the end of the contact phase. For each cytokine a preferred range of concentrations exists: IL-1β: 1-20 ng/ml, preferably 1-10 ng/ml, most preferably 10 ng/ml; IL-6: 50-200 U/ml, preferably 80-150 U/ml, most preferably 100 U/ml; TNF-α 1-20 ng/ml, preferably 1-10 ng/ml, most preferably 10 ng/ml; and PGE₂; 0.1-10 ng/ml, preferably 0.1-5 ng/ml, most preferably 1 ng/ml.

It is to be understood that the mature DCs of the invention generated in vitro are suitable for being administered (retransfused) to a patient.

Also an preferred aspect of embodiment (8) of the present invention is a method to generate a CD4⁺ T-cell clone specific for anyone of the MCSP peptides described above, the fusion proteins as described above, the full length MCSP protein of SEQ ID NO:1 and fragments thereof. Preferably, said cells are specific for anyone of the MCSP peptides or fusion proteins as described above or the full length MCSP protein, more preferably for the peptides or fusion proteins as described above. The preferred method comprises the following steps: (i) contacting isolated CD4⁺ T-cells with an antigen presenting cell (APC) presenting anyone of the peptides or proteins as described in the above paragraph, whereby the APC is selected from the group of B-lymphocytes, macrophages, and/or DCs. Preferentially the APC is a mature loaded DC generated by the method of the invention described above; (ii) co-culturing the isolated CD4⁺ T-cells with the APC for at least 30 days, whereby freshly prepared APCs are added for at least 3 times to the original co-culture, and the CD4⁺ T-cells proliferate; as culture medium is either RPMJ 1640 (Bio Whittaker) used, supplemented with 10% heat inactivated human pool serum, 2 mM L-glutamine and 20 μg/ml gentamicin; alternatively J-VIVO15 (Cumber) is used, which is supplemented analogously; (iii) assessing the ability of the proliferating CD4⁺ T-cells of (ii) to produce TNF-alpha and/or IFN-γ in response to the addition of stimulator cells pulsed with anyone of the peptides according to the invention, whereby the stimulator cells are selected from the group of autologous or allogenic immortalized B-cells, immortalized monocytes, macrophages or DCs, pulsed with anyone of the peptides according to the invention; autologous cells are preferred; (iv) cloning of the TNF-α and/or IFN-γ producing CD4⁺ T-cells of (iii) by limiting dilution culture in the presence of autologous or allogenic (autologous are preferred) stimulator cells pulsed with anyone of the peptides according to the invention, and feeder cells, whereby the feeder cells are selected from the group of allogenic or autologous immortalized B-cells such as LG2-EBV; and (v) maintaining the isolated CD4⁺ T-cell clone of step (iv) in the presence of feeder cells in culture medium comprising interleukin-2 (IL-2), interleukin-7 (IL-7) and phytohemagglutinin (PHA). The maintenance medium is selected from the group of RPMJ 1640 (Bio Whittaker) and J-VIVO 15 (Cumber) media, supplemented as described above with human serum, L-glutamine and gentamicin, wherein IL-2, IL-7 and PHA are present at concentrations ranging from 10 to 100 U/ml IL-2, preferably 50 U/ml, 1 to 10 ng/ml IL-7, preferably 5 ng/ml and 0.1 to 1 μg/ml PHA, preferably 0.5 μg/ml, respectively. Such a T-cell clone generated in vitro is suitable for in vivo transfer into a mammal, preferably into a human, for the prevention, treatment and/or diagnosis of cancer, preferably melanoma and other MCSP expressing tumours such as breast cancer, notably lobular breast carcinoma, astrocytoma, glioma, glioblastoma, neuroblastoma, sarcoma and certain types of leukaemia, in particular melanoma.

A preferred aspect of embodiment (9) of the present invention is a mature DC loaded with anyone of the peptides according to embodiment (1) or fusion proteins of embodiment (2) of the invention. Preferably the mature loaded DC is obtainable by the method according to the invention described above.

A preferred aspect of embodiment (10) is a CD4⁺ T-cell clone specific for anyone of the MCSP peptides of embodiment (1) or fusion proteins of embodiment (2). Preferred is a CD4⁺ T-cell clone obtainable by the method of embodiment (8) of the invention described above.

An antibody specific for anyone of the peptides of embodiment (1) described above is a preferred aspect of embodiment (11) of the present invention. Such antibodies can be produced by methods generally known in the art (see, e.g., Sambrook et al. in “Molecular Cloning. A Laboratory Manual”, Cold Spring Harbor Press, Plainview, N.Y. (1989)).

A preferred aspect of embodiment (12) is a composition comprising anyone of the MCSP peptides or fusion proteins according to embodiment (1) or (2) of the present invention, the nucleic acids according to embodiment (3) of the invention, the vectors according to embodiment (4) of the invention, the transformed/transfected cells according to embodiment (5) of the invention, the loaded mature DCs according to embodiment (9) of the invention, CD4⁺ and CD8⁺ T-cell clones according to embodiment (10) of the invention and/or antibodies according to embodiment (11) of the invention, and a pharmaceutically acceptable carrier. Said composition may be (and is preferably) a vaccine further comprising an adjuvant.

It is also an embodiment of the present invention to use anyone of the MCSP peptides or fusion proteins, the nucleic acid molecules and/or the vector molecules, described above for the preparation of immune cells, such as artificial APCs, mature DCs loaded with anyone of the peptides according to the invention, CD4⁺ and CD8⁺ T-cell clones specific for anyone of the peptides according to the invention, B-cells secreting antibodies specific for anyone of the MCSP peptides or fusion proteins according to the invention and/or hybridomas secreting antibodies specific for anyone of the MCSP peptides or fusion proteins according to the invention. Said preparation of immune cells may be performed ex vivo and in vivo. Ex vivo preparation is preferred.

A further embodiment is the use of anyone of the MCSP peptides according to the invention as a diagnostic marker for cancer, preferably as a diagnostic marker for melanoma and other MCSP expressing tumours such as breast cancer, notably lobular breast carcinoma, astrocytoma, glioma, glioblastoma, neuroblastoma, sarcoma and certain types of leukaemia, in particular for melanoma. The MCSP peptides according to the invention can be found on the cell surface of tumour cells, as well as on immune cells, if an immune reaction against the tumour is already raised. Thus, the MCSP peptides are suitable markers for in vivo and ex vivo imaging and/or detection of tumours.

Also an embodiment of the invention is the use of anyone of the MCSP peptides, the nucleic acid molecules and/or the vector molecules according to the invention, for the manufacturing of a medicament stimulating the production of protective antibodies and/or immune cells. Said medicament is particular useful for raising T-cell responses, especially CD4⁺ or CD8⁺ T cell responses. A consequence of administration of said medicament may be an immunization of the patient against the full length MCSP protein of SEQ ID NO:1 or fragments thereof.

Also an embodiment is the use of anyone of the MCSP peptides or fusion proteins, nucleic acid molecules, vector molecules, loaded mature DCs, CD4⁺ T-cell clones and/or the antibodies according to the invention, for the preparation of a medicament for preventing, treating and/or diagnosing cancer, preferably for preventing, treating and/or diagnosing melanoma, including cutaneous and ocular melanoma, and other MCSP expressing tumours such as breast cancer, notably lobular breast carcinoma, astrocytoma, glioma, glioblastoma, neuroblastoma, sarcoma and certain types of leukaemia, more preferably melanoma. Such a medicament may function as a vaccine.

Furthermore, embodiment (16) of the invention relates to an ex vivo method for diagnosing and/or monitoring a disorder characterized by the expression of anyone of the MCSP peptides according to the invention, comprising the following steps: (i) contacting a biological sample, such as a biopsy, isolated from a subject having or suspected to have said disorder, with an agent that is specific for anyone of the peptides according to the present invention, preferably the agent being a T-cell clone or an antibody according to the invention; and (ii) determining the interaction between the agent and the peptide. The term interaction refers here to a binding interaction, which triggers the T-cell clone to proliferate and to secrete cytokines, such as IFN-γ and/or TNF-α.

A final embodiment is a method for treating a subject having a disorder characterized by expression of anyone of the peptides according to the present invention alone, or as part of the MCSP, notably cancer. Said method comprises administering to the subject an amount of anyone of the peptides, the nucleic acid, the vector, the loaded mature DCs, the T-cell clone, the antibody, and/or anyone of the compositions, all according to the present invention. The amount to be administered will be determined by the medical practitioner individually for each patient and depends on various aspects including the type/severity of the disorder/cancer, the mode of administration, the age and weight of the patient, etc.

In the method of embodiment (17), the cancer is preferably selected from melanoma, including cutaneous and ocular melanoma, and other MCSP expressing tumours such as breast cancer, notably lobular breast carcinoma, astrocytoma, glioma, glioblastoma, neuroblastoma, sarcoma and certain types of leukaemia.

In a first trial to arrive at the peptides according to the invention, candidate peptides with 16 amino acids were chosen by screening for binding motifs for HLA-DP4 within the core protein of MCSP, and afterwards synthesized. HLA-DP-4 was selected because it is the most frequent HLA-class-II-molecule in Caucasians (expression by approx. 70%). Unfortunately, there are so far no complete algorithms to predict HLA-DP-4 binding motifs within a given protein sequence, as it already exists e.g. for HLA-DR-molecules. So the design of peptides was based on anchor motifs published before (amino acids F, L, Y or M for relative position 1; F, L, Y, A for position 7 and V, Y, I for position 10) or common sequences (e.g. MAGE-3.DP4-epitope). The following peptides were selected and synthesized: SQVLFSVTRGAHYGEL (SEQ ID NO:12), VRYLSTDPQHHAYDTV (SEQ ID NO:13), GEALVNFTQAEVYAGN (SEQ ID NO:14) and PHEVSVHINAHRLEIS (SEQ ID NO:15).

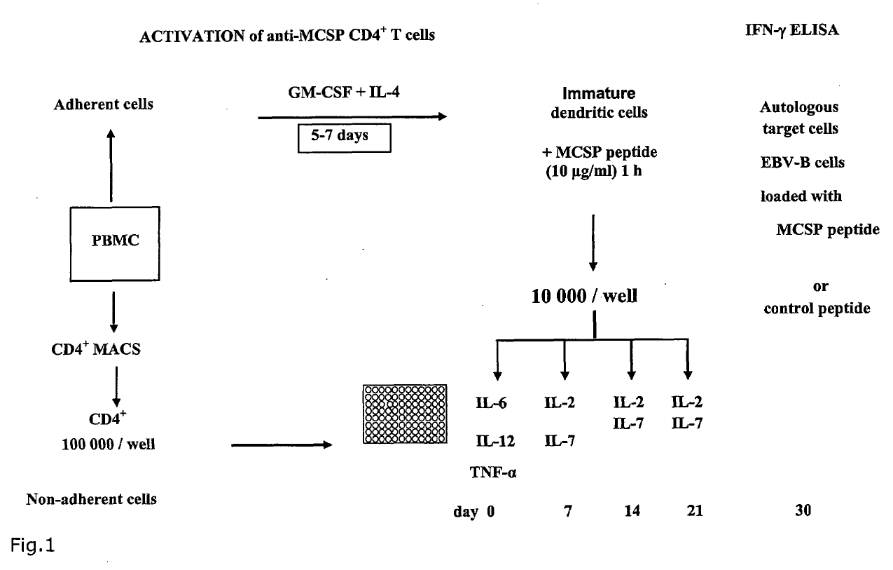

In order to test the ability of the above peptides to be T-cell stimulatory, DCs were derived from monocytes of healthy donors using IL-4 und GM-CSF and loaded with 10 μg/ml of candidate peptide for 1 hour. Autologous CD4⁺ T-cells were stimulated in 96-well-plates with those peptide-loaded DCs. After restimulation on day 7, 14 and 21 the existence of peptide-specific T-cells was tested by an ELISA technique. FIG. 1 shows schematically the experimental protocol. Although various experiments were performed, no peptide-specific CD4⁺ T-cells were induced. Obviously, the peptides with SEQ ID NO:12 to 15 do not represent naturally processed peptides recognized by T-cells. Accordingly the approach was changed to yield optimal benefit from the professional antigen processing and presentation by dendritic cells.

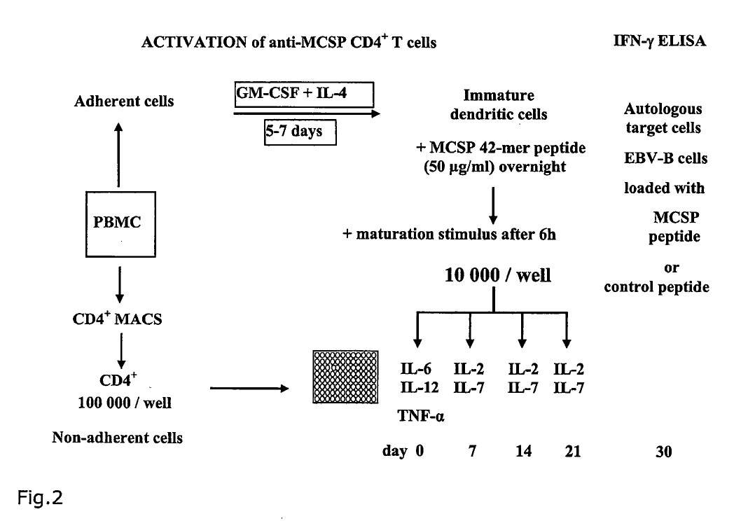

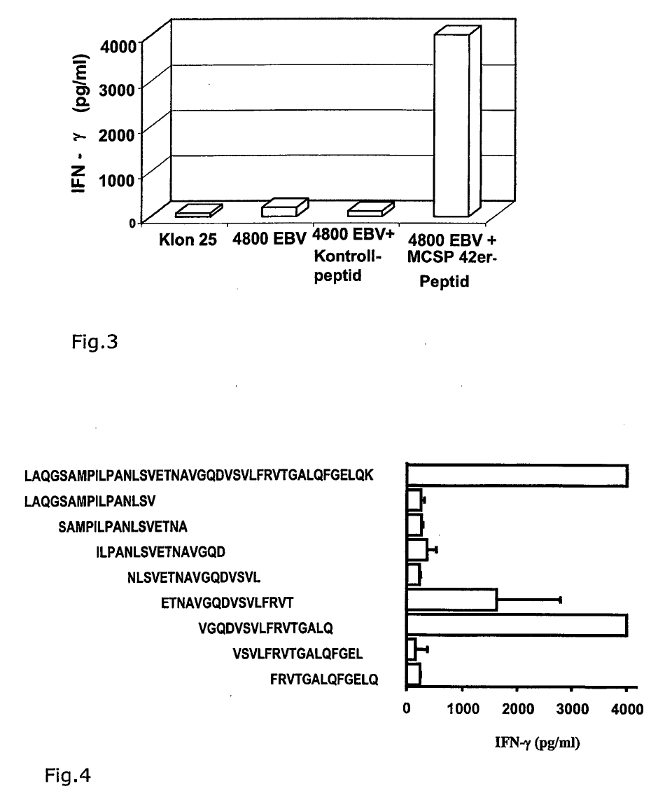

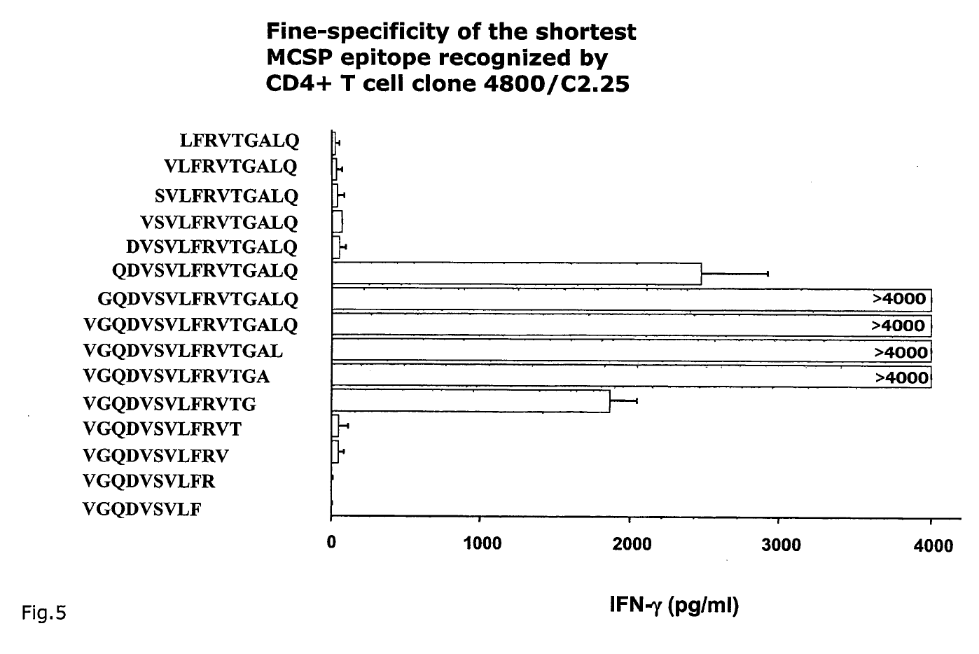

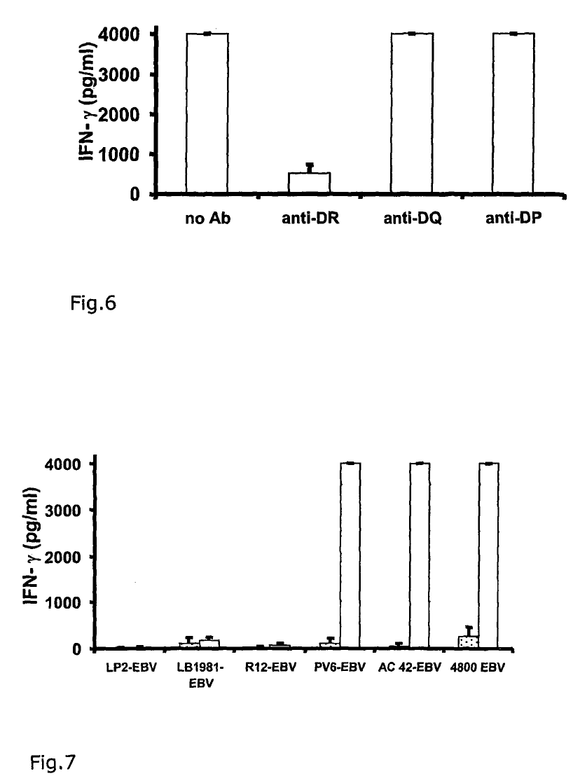

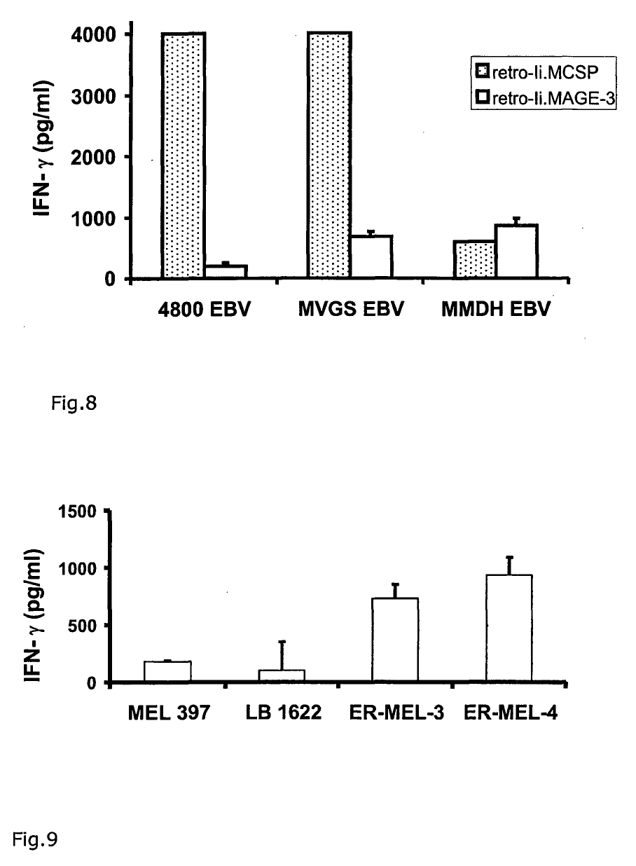

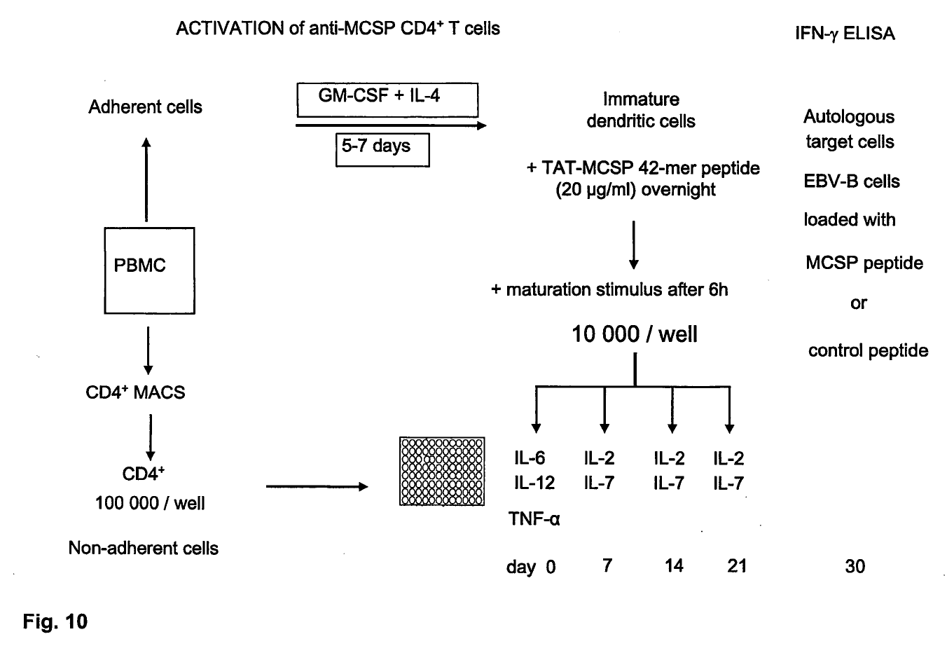

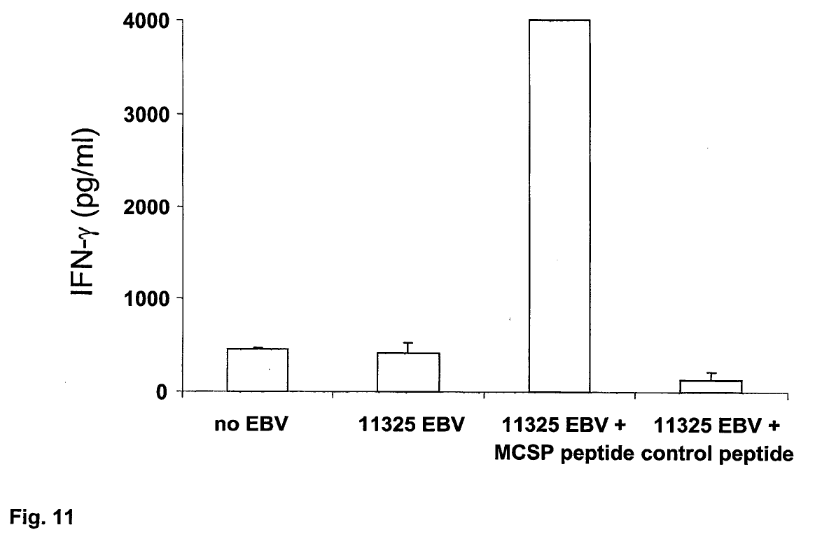

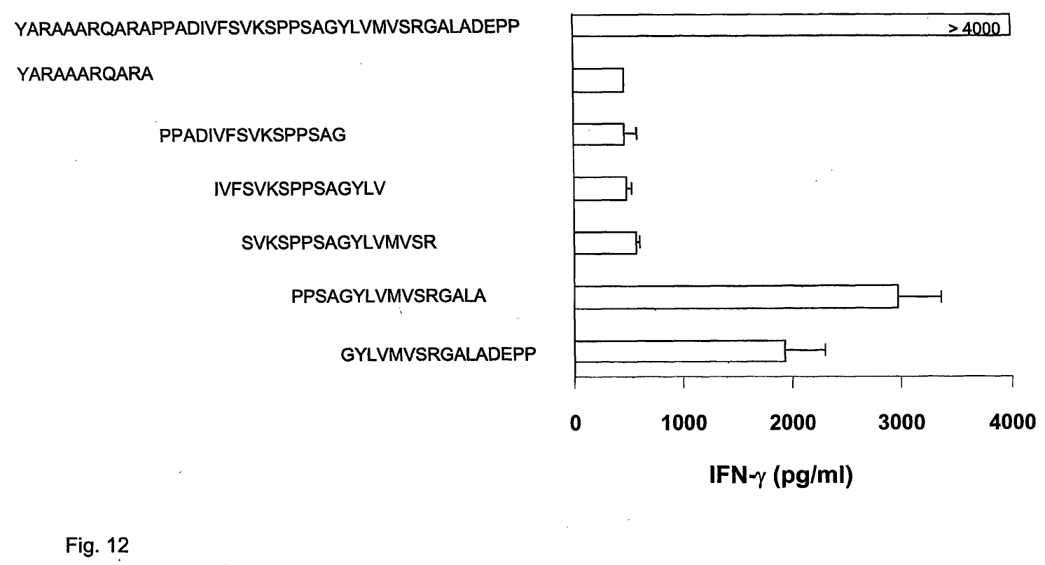

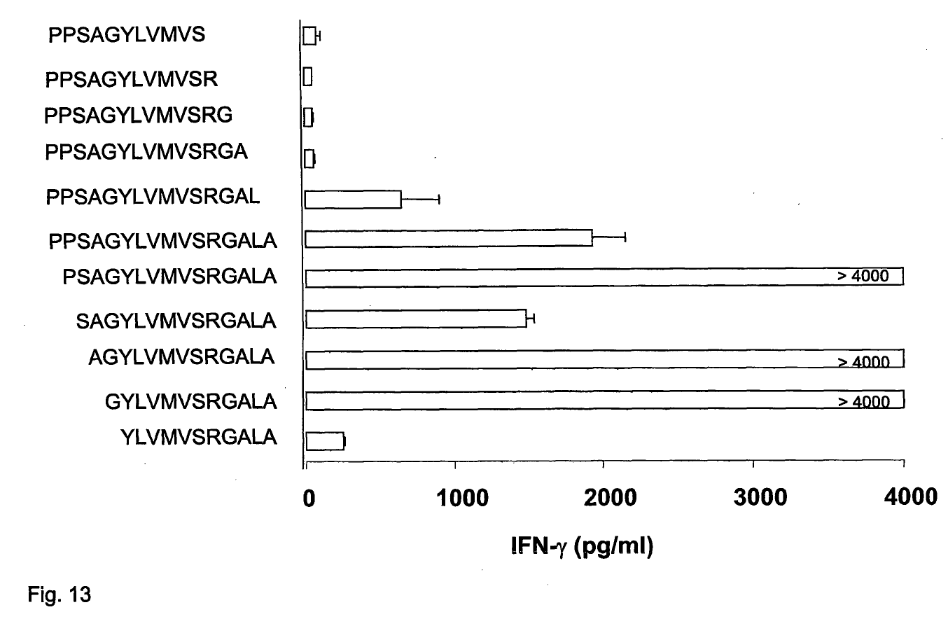

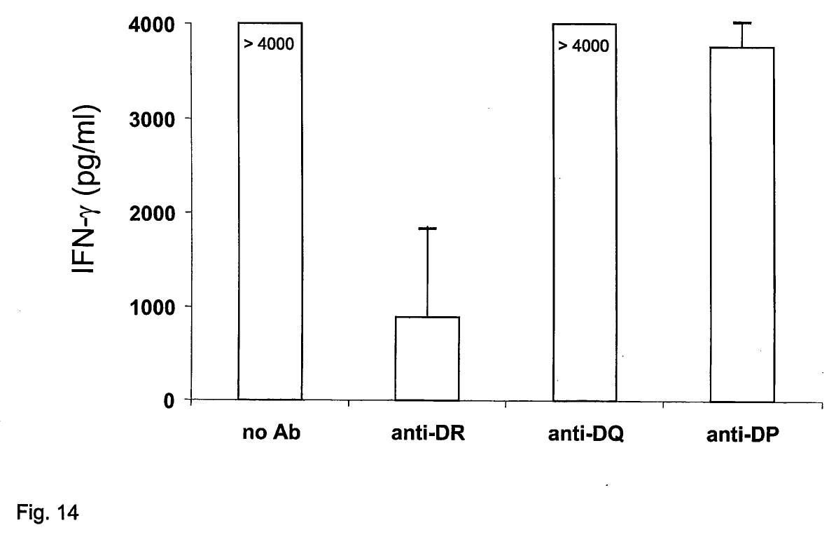

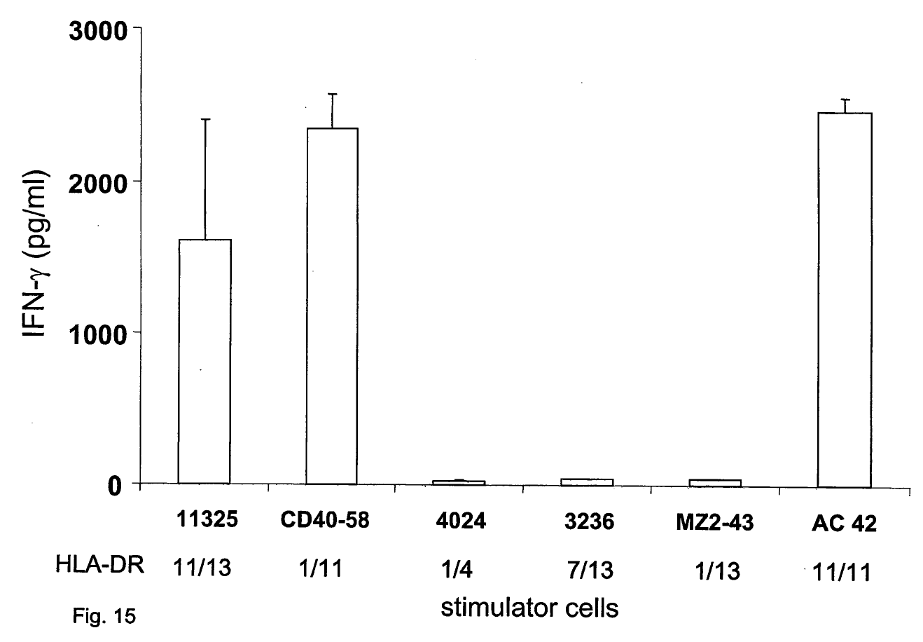

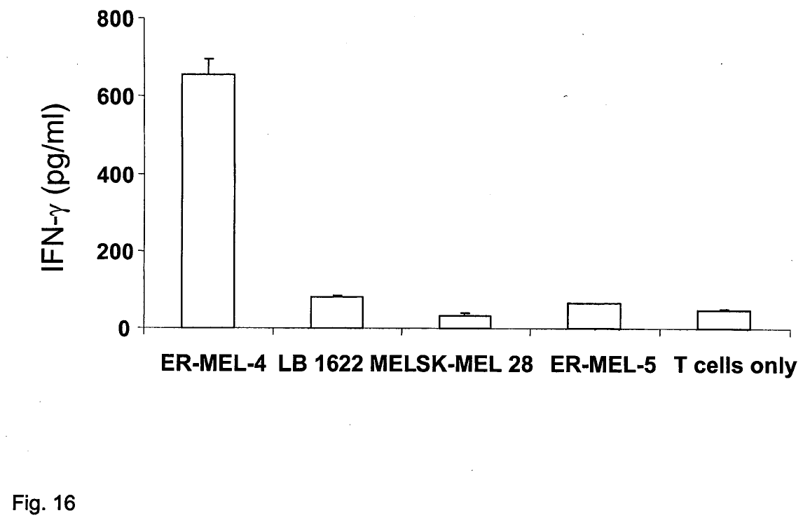

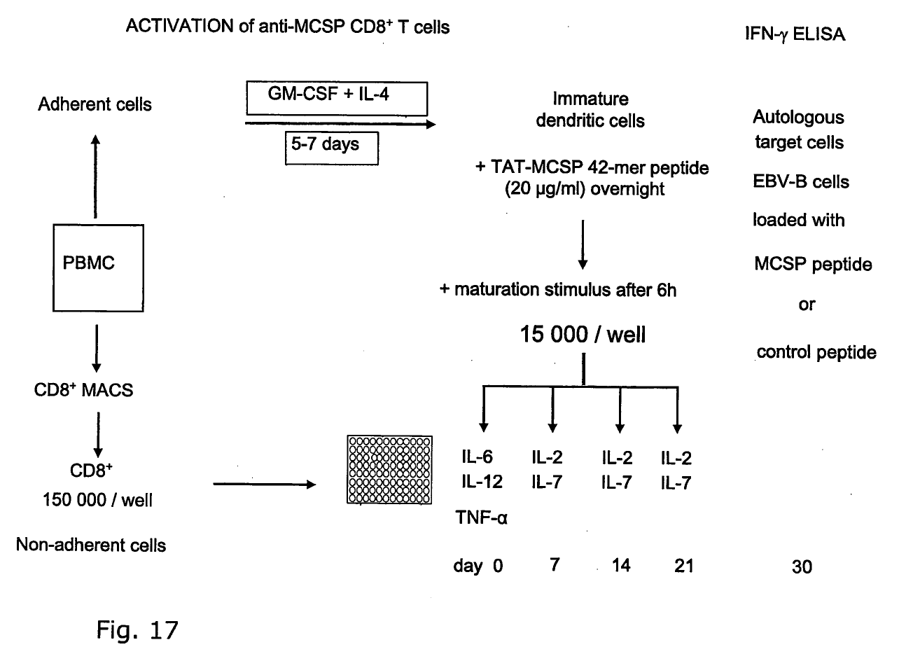

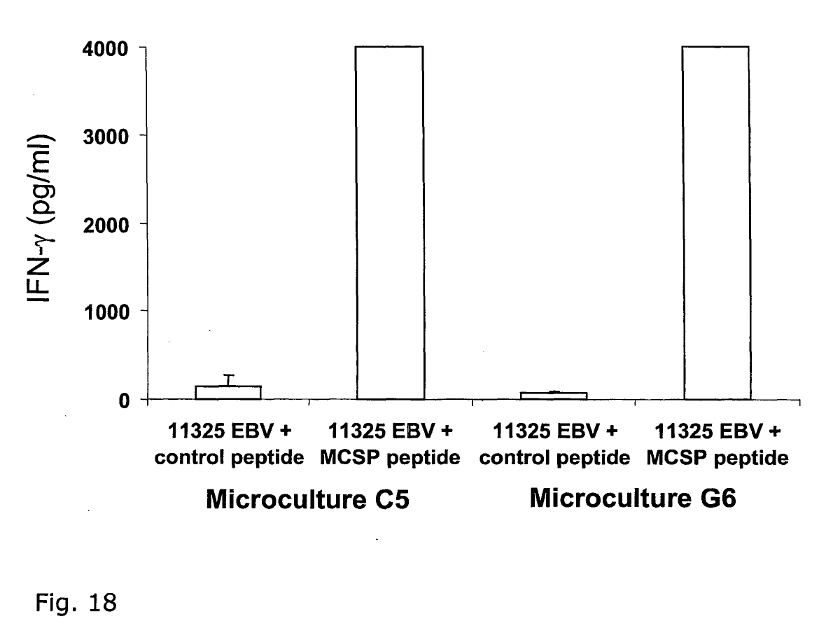

The complete MCSP core protein sequence was screened by computer algorithms (http://www.uni-tuebingen.de/uni/kxi, using the database SYFPEITHI), based on experimental cleavage data for the existence of peptides binding to different HLA-DR-molecules. A region was identified in which several HLA-DR-binding candidate antigens are localized, and a peptide with a length of 42 amino acids was synthesized. DCs were loaded with this long peptide overnight, so that potential T-cell epitopes within this peptide could be processed and presented by the cell. DCs were matured 6 h after peptide-loading by a cytokine cocktail (IL-1β, IL-6, TNF-alpha, PGE₂). CD4⁺ T-cells were stimulated with those protein-loaded mature DCs on the next day. FIG. 2 shows schematically the experimental protocol. Using this approach, 2 microcultures with peptide-specific CD4⁺ T-cells were generated and cloned by limiting dilution. In total, 3 peptide-specific T-cell clones were obtained from microculture C2. Further experiments were performed with clone C2/25, from hereon called in short C25. This clone showed a specific IFN-γ production after stimulation with peptide-loaded autologous EBV-B-cells, but not after stimulation with EBV-B-cells with or without a control peptide (FIG. 3).

To determine the core epitope recognized by clone 25 within the 42-mer, a set of 16-mer peptides, overlapping each other by 12 amino acids, was tested for recognition. Clone 25 recognized two overlapping 16-mer peptides, namely ETNAVGQDVSVLFRVT (SEQ ID NO:8) and VGQDVSVLFRVTGALQ (SEQ ID NO:9); (FIG. 4), but peptide VGQDVSVLFRVTGALQ (SEQ ID NO:9) was found to stimulate clone 25 most efficiently (FIG. 4). In a second step a set of truncated peptides derived from the sequence of the two overlapping peptides was tested to define the fine specificity of clone 25. The peptides were truncated for up to 7 amino acids at the N- or C-terminus. Truncations for up to 3 amino acids at both N- and C-terminus were tolerated. Truncations going beyond either D at amino acid position 696 (SEQ ID No. 1) at the N or the A at amino acid position 706 resulted in loss of recognition by the T cell clone (FIG. 5), as measured by an IFN-γ ELISA. Further titration experiments were carried out with the 16-mer MCSP-peptide VGQDVSVLFRVTGALQ (SEQ ID NO:9) (amino acids 693-708 of SEQ ID No. 1) and a concentration of 2 μg/ml was found optimal to achieve CD4⁺ T-cell stimulation. At 2 μg/ml of the peptide, the CD4⁺ T-cells secreted more than 4,000 μg/ml IFN-γ. This further confirmed that VGQDVSVLFRVTGALQ (SEQ ID NO:9) is very efficiently recognized by clone 25. Therefore this peptide was used in all further experiments.

To analyze the HLA restriction of clone 25, it was tested whether monoclonal anti-DR, anti-DQ or anti-DP antibodies would inhibit the recognition of antigen-presenting cells by the clone. The recognition of autologous stimulator EBV-B cells loaded with MCSP peptide VGQDVSVLFRVTGALQ (SEQ ID NO:9) (amino acids 693-708 of SEQ ID NO:1) was completely abolished by an anti-HLA-DR antibody, while antibodies against HLA-DP or HLA-DQ had no influence (FIG. 6), as determined by the IFN-γ production. Blood donor 4800, from whom the stimulator cells were derived, was typed HLA-DRB1*11/DRB1*03. In order to determine which allele clone 25 is restricted to, peptide VGQDVSVLFRVTGALQ (SEQ ID NO:9) was loaded on several EBV-B cell lines with different known HLA class II typings. In particular EBV-B cell lines LP2, LB1981-, R12-, PV6-, AC 42- and 4800-EBV were used, of which only PV6-, AC 42- and 4800-EBV are HLA-DR11 positive. It was found that only those expressing HLA-DRB1*11 (PV6-, AC 42- and 4800-EBV) were able to present the peptide to clone 25, as determined by IFN-γ production (FIG. 7). To establish that clone 25 reacted specifically with the MCSP peptide VGQDVSVLFRVTGALQ (SEQ ID NO:9) and its reaction was not caused by a contaminant within the peptide preparation or a chemical modification of the synthesized peptide, EBV-B cells stimulator cells were retrovirally transfected with the fusion peptide Ii comprising the amino acid residues 392-748 of MCSP, giving rise to the processed peptide VGQDVSVLFRVTGALQ (SEQ ID NO:9). For that, HLA-DR11 positive (4800- and MVGS EBV) and HLA-DR11 negative cells (MMDH EBV) were transduced with a retroviral construct retro-Ii.MCSP, which encodes a truncated human invariant chain (Ii) fused with a truncated MCSP (amino acid residues 392-748). The fusion guarantees an endosomal targeting and therefore effective processing and HLA class II presentation. Clone 25 reacted only with the recombinantly loaded HLA-DR11 positive cells, demonstrating that the clone was really MCSP-specific and not directed against a contaminant in the batch of the peptide VGQDVSVLFRVTGALQ (SEQ ID NO:9) (FIG. 8). From previous studies it was known, that tumour-specific CD4⁺ T cells can directly recognize HLA class II-expressing tumour cells. Therefore the ability of clone 25 to recognize directly MCSP-expressing melanoma cell lines was tested, as shown in FIG. 9. MCSP-positive melanoma cells expressing HLA-DRB1*11, namely ER-MEL-3 and ER-MEL-4, stimulated clone 25 to produce IFN-γ, whereas HLA-DR 11 negative cells, namely MEL 397, LB 1622, did not stimulate clone 25.

In conclusion, the aim set out at the beginning, namely to identify and characterize an MCSP-T-cell epitope was achieved. That way the hypothesis about the existence of MCSP-specific T-cell responses was impressively affirmed. MCSP-specific CD4⁺ T-cells were isolated and it was shown that endogenously processed antigen is recognized by said cells, which respond to the antigen by production of high amounts of IFN-γ. Moreover, it was demonstrated that the MCSP-specific CD4⁺ T-cells recognize tumour cells expressing MCSP. It is postulated therefore, that said cells play an important role in anti-tumour immunity in vivo. The identified peptide according to the invention can be used directly in immunotherapy against cancer, especially against malignant melanoma, in form of a medicament comprising said peptides. Said medicament may be used for peptide vaccination and/or for administering DCs loaded with anyone of the peptides according to the invention. It was shown here that MCSP and the peptide fragments derived therefrom are promising candidate antigens for immunotherapy, because they are expressed in >90% of melanoma. Considering the functional role of MCSP in growth, invasion and metastasis of malignant melanoma, a vaccination against MCSP could be useful, especially in an adjuvant setting after excision of larger primary tumours or regional lymph node metastasis. Phase I clinical studies involving vaccinating melanoma patients with the peptide-loaded DCs according to the invention are already planned.

In the passages above the identification of a MCSP CD4⁺ T helper cell epitope using dendritic cells loaded with a long candidate peptide as stimulator cells for CD4⁺ T cells was described. In further experiments another long peptide from the MCSP core protein sequence where computer algorithms had predicted several HLA-DR binding motifs and in addition a HLA-A2 binding motif was identified. This long peptide represented amino acids 1270-1300 (PPADIVFSVKSPPSAGYLVMVSRGALADEPP; SEQ ID NO:36) of MCSP. In order to identify both CD4⁺ and CD8⁺ T cell epitopes, said peptide was modified so that the peptide could enter the cytosol of the dendritic cells to get access to the HLA class I presentation pathway as follows: It has been shown that the protein transduction domain (PDT) embedded in the HIV TAT protein (amino acids 47-57=YGRKKRRQRRR; SEQ ID NO:52) can successfully mediate the introduction of peptides and proteins into mammalian cells in vitro and in vivo. Furthermore, it has been demonstrated that structurally modified nonnaturally occurring peptide variants can be even more efficient in protein transduction than the original wild type TAT PDT (Ho, A., et al., Cancer Research, 61: 474-477 (2001)). Therefore a TAT PDT variant peptide (YARAAARQARA; SEQ ID NO:53) was placed at the N-terminus of the above MCSP peptide resulting in a 42-mer with the following sequence: YARAAARQARAPPADIVFSVKSPPSAGYLVMVSRGALADEPP (SEQ ID NO:54) (“long TAT-MCSP-peptide”).

Dendritic cells (DC) were derived from monocytes of healthy donors using IL-4 and GM-CSF and loaded with the long peptide of SEQ ID NO:54 at 20 μg/ml overnight so that potential T-cell epitopes within this peptide could be processed and presented by the cell. DCs were matured 6 h after peptide-loading by a cytokine cocktail (IL-1β, IL-6, TNF-α, PGE₂) as described in Example 3 (b). Autologous CD4⁺ T-cells were stimulated with protein-loaded mature DCs in 96-well-plates on the next day (FIG. 10). After restimulation on day 7, 14 and 21 (compare Example 4) the existence of peptide-specific T-cells was tested by ELISA technique (compare Example 5). FIG. 10 shows schematically the experimental protocol.

3 microcultures with peptide-specific CD4⁺ T-cells could be generated and were cloned by limiting dilution (Example 5). 3 peptide-specific T-cell clones could be generated from microculture F1 in total. Further experiments were performed with clone F1/3. This clone showed a specific Interferon-γ production after stimulation with peptide-loaded autologous EBV-B-cells, but not after stimulation with EBV-B-cells with or without a control peptide (FIG. 11).

To determine the core epitope recognized by clone 3 within the 42-mer of SEQ ID NO:54, a set of overlapping peptides including the TAT PDT variant peptide was tested for recognition (Example 6). Clone 3 recognized two overlapping 16-mer peptides within the MCSP sequence but not the modified TAT peptide (FIG. 12). Peptide PPSAGYLVMVSRGALA (SEQ ID NO:42) was found to stimulate clone 3 most efficiently.

In a second step a set of truncated peptides derived from the sequence of peptide PPSAGYLVMVSRGALA (MCSP₁₂₈₁₋₁₂₉₆; SEQ ID NO:42) was tested to define the fine specificity of clone 3 (Example 7). Truncation of either G at the N terminus or L at the C-terminus resulted in loss of recognition by the T cell clone (FIG. 13). The 12-mer peptide GYLVMVSRGALA (MCSP₁₂₈₅₋₁₂₉₆; SEQ ID NO:46) turned out to be the shortest peptide efficiently recognized.

To analyze the HLA restriction of clone 3, it was tested whether monoclonal anti-DR, anti-DQ or anti-DP antibodies would inhibit the recognition of antigen-presenting cells by the clone (Example 8). The recognition by clone 3 of autologous EBV-B cells loaded with peptide MCSP₁₂₈₁₋₁₂₉₆ (SEQ ID NO:48) was abolished by an anti-HLA-DR antibody, while antibodies against HLA-DP or HLA-DQ had no influence (FIG. 14).

Blood donor 11325 was typed HLA-DR 11/13. To determine to which allele clone 3 is restricted to, peptide PPSAGYLVMVSRGALA (SEQ ID NO:42) was loaded on several EBV-B cell lines with different known HLA class II typings, and only those expressing HLA-DR 11 were able to present the peptide to clone 3 (FIG. 15; Example 9).

Since it has been shown that tumor-specific CD4⁺ T cells can directly recognize HLA class II-expressing tumor cells, the direct recognition of MCSP-expressing melanoma cell lines by clone 3 was assayed (Example 11). MCSP-positive melanoma cells expressing HLA-DR 11 (ER-MEL-4) stimulated clone 3 to produce IFN-γ, whereas HLA-DR 11 negative cells did not, as shown in FIG. 16.

In summary, using an approach with a TAT PDT variant peptide fused to the MCSP candidate peptide MCSP-specific CD4⁺ T cell clones were generated which recognize a different MCSP peptide presented by HLA-DR11 on human melanoma cells.

In order to isolate MCSP specific CD8⁺ T cells, DCs were derived from monocytes of healthy donors using IL-4 and GM-CSF and loaded with the long TAT-MCSP-peptide of SEQ ID NO:54 at 20 μg/ml overnight so that potential T-cell epitopes within this peptide could be processed and presented by the cell. DCs were matured 6 h after peptide-loading by a cytokine cocktail (IL-1β, IL-6, TNF-α, PGE₂) as described in Example 3 (b). Autologous CD8⁺ T-cells were stimulated with protein-loaded mature DCs in 96-well-plates on the next day (FIG. 17). After restimulation on day 7, 14 and 21 (compare Example 4) the existence of peptide-specific T-cells was tested by ELISA technique (compare Example 5). FIG. 17 shows schematically the experimental protocol.

Furthermore, CD8⁺ T-cells from microcultures showed a specific IFN-γ production after stimulation with autologous EBV-B-cells of donor 11325 loaded with the long TAT-MCSP-peptide (SEQ ID NO:54; 10 μM), but not after stimulation with EBV-V cells with a control peptide (FIG. 18; Example 5).

The invention is further described by the following examples which are, however, not to be construed as to limit the invention.

### EXAMPLES

1. Computer Algorithms: The complete MCSP core protein sequence was screened by computer algorithms (http://www.uni-tuebingen.de/uni/kxi, using the database SYFPEITHI). Various HLA I/II alleles were chosen, inter alia DR 0101, DR 0301, DR 0401, DR 0701, DR 1101, DR 1501. Candidate peptides were predicted for the various DR molecules.

2. Peptid synthesis: Peptides were synthesized using F-moc for transient NH₂-terminal protection and were characterized using mass spectrometry. All peptides were >80% pure as indicated by analytical HPLC. Lyophilized synthetic peptides were dissolved in DMSO (Merck)/acetic acid (10 mM) and stored at −20° C. Peptides were purchased from Coring System Diagnostix GmbH (Gernsheim, Germany).

### 3. Preparation of Peptide Loaded Mature DCs and CD4⁺ T-Cells:

(a) Isolation of immature DCs and CD4⁺ T-responder cells: Peripheral blood mononuclear cells (PBMCs) were isolated from leukapheresis products obtained from healthy donors after informed consent. First, PBMCs were isolated by centrifugation on Lymphoprep (Nycomed Pharma, Oslo, Norway). In order to minimize contamination of PBMCs with platelets, the preparation was first centrifuged for 20 min/1,000 rpm at room temperature. After removal of the top 20-25 ml, containing most of the platelets, the tubes were centrifuged for 20 min/1,500 rpm at room temperature. The interphase containing the PBMCs was harvested and then 3× washed (or more) in cold phosphate buffer solution with 2 mM EDTA in order to eliminate any remaining platelets.

PBMCs were plated in 85 mm tissue culture dishes (Falcon. Cat. No. 3003; Becton Dickinson, Hershey, USA) at a density of 50×10⁶ cells per dish in 10 ml RPMI 1640, supplemented with 1% heat-inactivated autologous plasma, 2 mM L-glutamine and 20 μg/ml gentamicin, hereafter referred to as complete DC medium, and incubated at 37° C. and 5% CO₂ for 2 h. The non-adherent fraction was removed and frozen and 10 ml of complete DC medium was added to the adherent cells. On day 1 and 3 1,000 U/ml GM-CSF and 800 U/ml IL-4 (both from CellGenix) were added to the cultures to induce the differentiation of the adherent monocytes. On day 5 the non-adherent cells were used as a source of enriched immature DCs. Immature DCs were then loaded with the MCSP 42-mer peptide (either SEQ ID NO:3 or SEQ ID NO:54) at 50 μg/ml (SEQ ID NO:3) or 20 μg/ml (SEQ ID NO:54) overnight in complete DC medium and after 6 h maturation was induced by adding IL-1β (10 ng/ml), IL-6 (100 U/ml), TNF-α (10 ng/ml) and PGE₂ (1 μg/ml) to the culture medium (see also below under (b)). The next day the non-adherent fraction was thawed and CD4⁺ T lymphocytes were isolated by positive selection using an anti-CD4 monoclonal antibody coupled to microbeads (Miltenyi Biotech, Bergisch Gladbach, Germany) (for more details see section above).

(b) Peptide loading/pulsing of immature DCs and maturation: Immature DCs (1×10⁵) were incubated at 37° C., 5% CO₂, for 1 h or 20 h (overnight) in RPMI medium supplemented with 1% human serum, IL-4 (100 U/ml) and GM-CSF (100 ng/ml) and TNF-α (1 ng/ml) in the presence of MCSP-derived peptides or control peptides at concentrations of between 2-50 μg/ml. In case the DCs were incubated overnight, the DCs received a maturation stimulus after 6 h by addition of IL-1β (10 ng/ml), IL-6 (100 U/ml), TNF-α (10 ng/ml) and PGE₂ (1 μg/ml) to the culture medium. The pulsed/loaded DCs were washed and subsequently used in the stimulation assay.

### 4. T-Cell Induction Assay:

(A) CD4⁺ T-cell induction: On the day of stimulation (day 0) loaded DCs were washed and added at 1×10⁴ per round-bottom microtiter dish well to 10⁵ CD4⁺ T-cells in 200 μl complete T-cell medium in the presence of IL-6 (1000 U/ml), IL-12 (10 ng/ml) and TNF-α (1 ng/ml). The CD4⁺ T-cells were weekly (days 7, 14 and 21) restimulated with autologous DCs freshly loaded/pulsed with the MCSP peptide and were grown in complete T-cell medium supplemented with IL-2 (10 U/ml), IL-7 (5 ng/ml).

(B) CD8⁺ T cell induction: On the day of stimulation (day 0) loaded DCs were washed and added at 1.5×10⁴ per round-bottom microtiter dish well to 1.5×10⁵ CD8⁺ T-cells of donor 11325 in 200 μl complete T-cell medium in the presence of IL-6 (1000 U/ml), IL-12 (10 ng/ml) and TNF-α (1 ng/ml). The CD8⁺ T-cells were weekly (days 7, 14 and 21) restimulated with autologous DCs freshly loaded/pulsed with the MCSP peptide and were grown in complete T-cell medium supplemented with IL-2 (10 U/ml), IL-7 (5 ng/ml).

5. IFN-γ production by stimulated CD4⁺ and CD8⁺ T cells; Obtention of CD4⁺ T-cell lines and clones specific for the MCSP peptide: The microcultures of stimulated CD4⁺ or CD8⁺ T-cells (see Example 4) were assessed on day 30 after start of the culture for their capacity to produce IFN-γ when stimulated with autologous EBV-B cells pulsed/loaded with the MCSP peptide. Autologous EBV-B cells (5×10⁵) were incubated for 18-20 h at 37° C. in the presence of 10 μg/ml or 20 μg/ml (compare Figure legends) of the MCSP peptide or an irrelevant control peptide as a negative control. EBV-B-cells referred to herein are B-cells which were immortalized with Epstein Barr virus. The EBV-B-cells were prepared according to art-standard procedures. Peptide-pulsed EBV-B cells were washed and added at 1.5×10⁴ cells per round-bottom well to 4×10³ CD4⁺ or CD8⁺ T-cells in 100 μl of complete T-cell medium supplemented with IL-2 (25 U/ml). After 18-20 h, supernatants were harvested and assessed for IFN-γ content using an ELISA assay with reagents from Medgenix Diagnostics-Biosource (Fleurus, Belgium). Briefly the assay is a standard ELISA in which IFN-γ antibodies were coated onto the wells of plastic microtiter plates prior to incubation with cell supernatants to determine the amount of IFN-γ produced, with a specificity of 20-4,000 pg/ml. Cytokine secretion was considered significant if it was at least two-fold above the background response of T-cells to EBV-B cells pulsed/loaded with the control peptide, and if it exceeded 500 pg of IFN-γ per ml. Often the IFN-γ production was found to overshoot the upper specificity range of the ELISA, and then the amount of IFN-γ was said to be >4,000 μg/ml.

The CD4⁺ T-cell lines producing IFN-γ, i.e. those which recognize the MCSP peptide, were cloned by limiting dilution. Three CD4⁺ T-cell clones were obtained and grown in complete T-cell medium supplemented with IL-2 (50 U/ml), IL-7 (5 ng/ml). The clones were supplemented with fresh culture medium once a week and passaged with EBV-B stimulator cells pulsed/loaded with the MCSP peptide at 1-2 week intervals. Clone C2/25 was selected for all further experiments on the peptide with SEQ ID NO:3. Further experiments on the peptide with SEQ ID NO:54 were performed with clone F1/3.

6. Identification of MCSP HLA-DR restricted peptide: In order to identify the core epitope recognized by CD4⁺ T-cell clone C2/25 (“25”), 14 to 16 amino acid peptides (SEQ ID NOs:3-10) corresponding to overlapping parts of the MCSP peptide with SEQ ID NO:3 were synthesized and loaded onto autologous EBV-B cells and tested for recognition (FIG. 4).

To determine the core epitope recognized by clone F1/3 (“3”) within the 42-mer of SEQ ID NO:54, a set of overlapping peptides including the TAT PDT variant peptide was tested for recognition (FIG. 12).

Peptides were synthesized using F-moc for transient NH₂-terminal protection and were characterized using mass spectrometry. All peptides were >80% pure as indicated by analytical HPLC. Lyophilized synthetic peptides were dissolved in DMSO (Merck)/acetic acid and used at a final concentration of 1 μM/ml. EBV-B cells (1.5×10⁴ per round-bottomed microtiter plate well) were incubated at 1 h at 37° C., 8% CO₂ in the presence of the different peptides. The CD4⁺ T-cell clone 25 or 3, respectively, was then added at 4×10³ per well. Assay medium was complete T-cell medium supplemented IL-2 (25 U/ml). After 18-20 h, supernatants were harvested and assessed for IFN-γ production using an ELISA assay.

The two overlapping peptides ETNAVGQDVSVLFRVT (SEQ ID NO:8) and VGQDVSVLFRVTGALQ (SEQ ID NO:9) were found to stimulate clone 25, whereby the peptide with SEQ ID NO:9 had the most stimulatory effect. Clone 3 recognized two overlapping 16-mer peptides within the MCSP sequence but not the modified TAT peptide (FIG. 12). Peptide PPSAGYLVMVSRGALA (SEQ ID NO:42) was found to stimulate clone 3 most efficiently.

7. Determination of minimal peptides still able to stimulate CD4⁺ T-cell clone 25 or 3: Unlike HLA class I restricted peptides, class II peptides vary considerably in length and can tolerate truncation and extension at both N- and C-termini. Therefore the ability of truncated peptides (LFRVTGALQ (SEQ ID NO:22), VLFRVTGALQ (SEQ ID NO:23), SVLFRVTGALQ (SEQ ID NO:24), VSVLFRVTGALQ (SEQ ID NO:25), DVSVLFRVTGALQ (SEQ ID NO:26), QDVSVLFRVTGALQ (SEQ ID NO:27), GQDVSVLFRVTGALQ (SEQ ID NO:28), VGQDVSVLFRVTGAL (SEQ ID NO:29), VGQDVSVLFRVTGA (SEQ ID NO:30), VGQDVSVLFRVTG (SEQ ID NO:31), VGQDVSVLFRVT (SEQ ID NO:32), VGQDVSVLFRV (SEQ ID NO:33), VGQDVSVLFR (SEQ ID NO:34), VGQDVSVLF (SEQ ID NO:35) to stimulate the CD4⁺ T-cell clone 25 was compared to the untruncated peptide VGQDVSVLFRVTGALQ (SEQ ID NO:9). In essence EBV-B cells (5×10³ per round-bottomed microtiter plate well) were incubated at 2 h at 37° C., 8% CO₂ in the presence of the different peptides. The CD4⁺ T-cell clone 25 was then added at 2.5×10³ per well. Assay medium was complete T-cell medium supplemented with IL-2 (25 U/ml). After 18-20 h, supernatants were harvested and assessed for IFN-γ production using an ELISA assay. The results are summarized in FIG. 5.

Analogously, a set of truncated peptides derived from the sequence of peptide PPSAGYLVMVSRGALA (MCSP₁₂₈₁₋₁₂₉₆; SEQ ID NO:42) was tested to define the fine specificity of clone 3. Truncation of either G at the N terminus or L at the C-terminus resulted in loss of recognition by the T cell clone (FIG. 13). The 12-mer peptide GYLVMVSRGALA (MCSP₁₂₈₅₋₁₂₉₆; SEQ ID NO:46) turned out to be the shortest peptide efficiently recognized.

8. Determination of HLA restriction element utilized by MCSP-specific CD4⁺ T-cell clone 25 and 3: To analyze the HLA restriction of CD4⁺ T-cell clone 25 and 3, three monoclonal antibodies directed against anti-DR (L243; BD Biosciences, San Jose, USA), anti-DQ (CSPVL3; Immunotech) or anti-DP (B7/21; BD Biosciences, San Jose, USA) were used to test which one inhibited the recognition of antigen-presenting cells by the clone. First EBV-B cells (1.5×10⁴ per round-bottomed microtiter plate well) were incubated for 1 h at 37° C., 8% CO₂ in the presence of the peptide (SEQ ID NO:9 for clone 25, SEQ ID NO:48 for clone 3). The antibodies were added at a concentration of 5 ng/ml. The CD4⁺ T-cell clone 25 or 3 was then added at 4×10³ per well. Assay medium was complete T-cell medium supplemented with IL-2 (25 U/ml). After 18-20 h, supernatants were harvested and assessed for IFN-γ production using an ELISA assay.

9. Determination of HLA-DR restriction element utilized by MCSP-specific CD4⁺ T-cell clone 25: It was determined that cytokine secretion by the CD4⁺ T-cell clone 25 occurred in response to EBV-B cells pulsed/loaded with MCSP-derived peptides restricted to HLA-DR. To further define the HLA-restriction element utilized by clone 25, additional EBV-B cell lines were used for peptide presentation as described above. In particular LP2-, LB1981-, R12-, PV6-, AC 42- and 4800-EBV which have with different HLA class II molecules (HLA-DR11 positive or HLA-DR11 negative) were used. They were pulsed/loaded for 1 h with 1 μM MCSP peptide VGQDVSVLFRVTGALQ (SEQ ID NO:9) and tested for recognition by the CD4⁺ T cell clone. As control, the cells were pulsed in the absence of protein. IFN-γ production of the CD4⁺ T cells was measured after overnight co-culture (20 h) by ELISA. The results are summarized in FIG. 7.

10. Preparation and use of MCSP peptide fusion protein: An EBV-B cell line was transduced with the retroviral construct retro-Ii.MCSP, which encodes a truncated human invariant chain (Ii) fused with a truncated MCSP (amino acid residues 392-748). The construct was kindly provided by Professor Gerd Pluschke, Swiss Tropical Institute, Basel, Switzerland.

(a) Plasmids and cloning of fusion constructs: The MCSP cDNA and its corresponding polypeptide are set forth in SEQ ID Nos:1 and 2, respectively. The plasmid comprising the human invariant chain (Ii) cDNA, named lipSV51L, was kindly provided by Dr. J. Pieters (Basel Institute for Immunology, Basel, Switzerland; J. Cell Science 106, 831-846 (1993)). The plasmid pMFG was kindly provided by Dr. O. Danos (Somatrix Therapy Corporation, Alameda, Calif. USA) The MCSP cDNA coding for amino acid residues 659-735 (SEQ ID NO:1) was transferred into the pMFG vector after introduction of appropriate restriction enzyme recognition sites at the 5′ and 3′ end of the coding sequence. A BamHI restriction site was introduced at the 5′ end and at the 3′ end, by PCR using the forward primers 5′-GGGGATCCCATCCGGCCGGCCATACAG-3′ (SEQ ID NO:16) and the reverse primer: 5′-GGGGATCCTCACCGCTGGTGGAACGCCTGTG-3′ (SEQ ID NO:17); the BamHI restriction is shown in italic, and the stop codon is underlined. The PCR product was cloned into a pCR2.1 vector and sequenced according to standard methods. The BamHI-BamHI amplification product was cloned into pMFG opened with the enzyme BamHI, to give rise to pMFG-MCSP.

The cDNA encoding the amino terminal end (i.e. the cytoplasmic tail and the transmembrane region) of the human invariant chain polypeptide (hu-Ii; residues 1-80) was amplified by PCR using IipSV51L as template. The following primers were used: hu-Ii sense: 5′-TTTCCATGGATGACCAGCGCGAC-3′ (SEQ ID NO:18); and hu-Ii antisense: 5′-TTTGGATCCGGAAGCTTCATGCGCAGGTTC-3′ (SEQ ID NO:19); The recognition sites for NcoI and Bam HI are in italic. The PCR product was cloned into pCR2.1 and sequenced according to standard methods. The NcoI and BamHI amplification product was cloned into pMFG, opened with the enzymes NcoI and BamHI resulting in pMFG-Ii.

The recombinant plasmid pMFG-Ii was reopened with BamHI, and the BamHI MCSP fragment isolated from pMFG-MCSP ligated. The resulting plasmid gives rise to the fusion protein huIi.MCSP (in short Ii-MCSP or Ii). Recombinant plasmids containing the MCSP fragment in the right orientation were identified by restriction fragment analysis.

The DNA sequence of huIi-MCSP (SEQ ID NO:20) (start and stop codons are in bold; the huIi fragment is in small letters; the MCSP fragment is in upper case; a detected sequence variation is underlined; there the sequence differs from the published sequence (TAGTA becomes GGGGG; since the sequence variation lies outside the target area, experiments were continued with said construct):

atggaccttatctccaacaatgagcaactgcccatgctgggccggcgccc
tggggccccggagagcaagtgcagccgcggagccctgtacacaggctttt
ccatcctggtgactctgctcctcgctggccaggccaccaccgcctacttc
ctgtaccagcagcagggccggctggacaaactgacagtcacctcccagaa
cctgcagctggagaacctgcgcatgaagcttcccaaggatcccATCCGGC
CGGCCATACAGATCCACCGCAGCACAGGGTTGCGACTGGCCCAAGGCTCT
GCCATGCCCATCTTGCCCGCCAACCTGTCGGTGGAGACCAATGCCGTGGG
GCAGGATGTGAGCGTGCTGTTCCGCGTCACTGGGGCCCTGCAGTTTGGGG
AGCTGCAGAAGCAGGGGGCAGGTGGGGTGGAGGGTGCTGAGTGGTGGGCC
ACACAGGCGTTCCACCAGCGGTGA.

The corresponding amino acid sequence is as follows (SEQ ID NO:21) (the huIi fragment is in small letters; the MCSP fragment is in upper case; the detected sequence variation is underlined; there the sequence differs from the published sequence (HST becomes QGA; since the sequence variation lies outside the target area, experiments were continued with said construct):

mdlisnneqlpmlgrrpgapeskcsrgalytgfsilvtlllagqattayf
lyqqqgrldkltvtsqnlqlenlrmklpkdpIRPAIQIHRSTGLRLAQGS
AMPILPANLSVETNAVGQDVSVLFRVTGALQFGELQKQGAGGVEGAEWWA
TQAFHQRZ.

(b) Retroviral transduction of EBV cell lines: EBV-transformed B cells with different HLA class II molecules (4800 EBV and MVGS EBV as HLA-DR11 positive cells and MMDH EBV as HLA-Dr11 negative cells) were infected by resuspending the cells in an infection cocktail and centrifugation. Target cells were resuspended in 60 mm tissue culture plates (Falcon) at a density of 1×10⁶ cells in 4 ml infection cocktail. The plates were centrifuged for 2 h at 32° C. and 1,200 rpm in an ICE centrifuge, rotor type 228. for each plate to be transduced, 4 ml of infection cocktail was prepared by diluting the viral supernatant 1:2 in EBV B-cell growth medium and adding protamine sulfate to a final concentration of 6 μg/ml. Centrifugation was followed by another 2 h of incubation in a humidified incubator at 37° C. and cells were transferred to 4 ml of target cell growth medium. This transduction cycle was carried out immediately after plating the cells and was repeated at 24 h and 48 h. The infected EBV-B cells were assayed for EGFP reporter gene expression by FACS analysis 24 h to 48 h following the third infection cycle.

(c) IFN-γ production assay: 4×10³ CD4⁺ T-cells of clone 25 were washed and cultured overnight in the presence of 5×10³ retrovirally transduced EBV B-cells (4800 EBV, MVGS EBV and MMDH EBV), in 100 μl complete T-cell medium containing 25 U/ml recombinant human IL-2 in a round-bottom 96 well plate. All co-cultures were performed in triplicate. 50 μl culture supernatant was assayed for the presence of IFN-γ by ELISA (IFN-γ ELISA Biosource). Briefly, ELISA plates precoated with anti-human IFN-γ antibodies were washed and incubated with 50 μl of culture supernatant and 50 μl of biotinylated anti-human IFN-γ antibody (1:1,250) for 2 h at room temperature. Following three washes the plates were incubated with 50 μl per well horseradish peroxidase conjugated streptavidin (1:3,000 in PBS with 0.5% BSA) for 30 min at room temperature, which was detected by TMB substrate, and H₂SO₄ to stop the reaction. The optical density was read at 450 nm. Samples containing 4,000 μg/ml IFN-γ and 1:2 dilutions were used as standards.

11. MCSP positive melanoma cells as stimulators for CD4⁺ T-cell clone 25 and 3: In order to test whether the CD4⁺ T-cell clones 25 and 3 can directly recognize HLA-DR11 positive melanoma cells, melanoma cells which were known to be either HLA-DR11 positive or negative (MEL 397 (HLA-DR11 negative), LB 1622 (HLA-DR11 negative), ER-MEL-3 (HLA-DR11 positive) and ER-MEL-4 (HLA-DR11 positive) were preincubated at 2×10⁴ cells in flat bottom microwells for 24-48 h to reach a monolayer of tumour cells. Clone 25 or 3, respectively, (4×10³ cells/well) was then added and after co-culturing for 20 h the supernatant was tested for the presence of IFN-γ by ELISA. The results are shown in FIGS. 9 and 16.

1-23. (canceled)

24. An antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof.

25. The MCSP peptide of claim 24, which comprises at least 12 amino acid residues.

26. The MCSP peptide of claim 24, which is presented by a molecule selected from the group consisting of HLA-DR, HLA-DQ and HLA-DP.

27. The MSCP peptide of claim 26, which is presented in full length by said molecule.

28. The MSCP peptide of claim 26, which is presented in fragmented form by said molecule.

29. The MCSP peptide of claim 26, which is HLA-DR presented.

30. The MCSP peptide of claim 24, which comprises the amino acid residues 695 to 705 of SEQ ID NO:1.

31. The MCSP peptide of claim 24, which is selected from the group consisting of peptides having the following amino acid sequences
(a) LAQGSAMPILPANLSVETNAVGQDVSVLFRVTGALQFGELQK (SEQ ID NO:3);
(b) ETNAVGQDVSVLFRVT (SEQ ID NO:8),
(c) VGQDVSVLFRVTGALQ (SEQ ID NO:9),
and fragments of SEQ ID NOs:3, 8 or 9 shortened by up to three C-terminal and/or N-terminal amino acids.

32. The MCSP peptide of claim 31, which is selected from the group consisting of SEQ ID NOs:3, 8 and 9 being not shortened on its termini.

33. A fusion protein comprising as a first domain an MCSP peptide according to claim 24 (MCSP domain) and at least one second domain.

34. The fusion protein of claim 33, wherein said at least one second domain is a protein and/or peptide comprising an endosomal targeting signal.

35. The fusion protein of claim 33, wherein said at least one second domain is selected from the group of the human invariable chain (Ii), a peptide fragment thereof comprising amino acid residues 1-80, the lysosome-associated membrane protein (LAMP-1) and DC-LAMP.

36. The fusion protein of claim 33, wherein said MCSP domain and said at least one second domain are connected directly or through a linker peptide.

37. A nucleic acid sequence encoding the MCSP peptide of claim 24.

38. A nucleic acid sequence encoding the fusion protein of claim 33.

39. A vector comprising the nucleic acid sequence of claim 37.

40. A vector comprising the nucleic acid sequence of claim 38.

41. A cell transfected or transformed with the vector of claim 39, and/or comprising a nucleic acid encoding an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof.

42. A cell transfected or transformed with the vector of claim 40, and/or comprising a nucleic acid encoding a fusion protein comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain.

43. A method to generate stable mature dendritic cells (DCs) loaded with one or more of the peptides/proteins selected from the group consisting of (a) one or more of the MCSP peptides and functional variants of claim 24, (b) one or more fusion proteins comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain., and (c) the full length MCSP protein of SEQ ID NO:1 and fragments thereof, which method comprises the following steps:
(i) contacting isolated immature DCs or mature DCs with one or more of said peptides/proteins (a) to (c) defined above, to allow for uptake of said peptides/proteins, or contacting isolated immature DCs or mature DCs with one or more of the nucleic acid sequence encoding the peptides/proteins (a) to (c) defined above and/or with one or more vectors comprising nucleic acid sequences encoding the peptides/proteins (a) to (c) defined above, to allow for uptake and subsequent expression of the peptides/proteins in the DC; and
(ii) in case of immature DCs, maturing the DCs obtained in (i) by exposing them to a cytokine comprising maturation cocktail.

44. A method to generate a T-cell clone specific for one or more of the peptides/proteins selected from the group consisting of (a) one or more of the MCSP peptides and functional variants of claim 24, and (b) the full length MCSP protein of SEQ ID NO:1 and fragments thereof, which method comprises the following steps:
(i) contacting isolated T-cells with an antigen presenting cell (APC) presenting anyone of the peptides/proteins (a) to (b) as defined above, whereby the APC is selected from the group consisting of B-lymphocytes, macrophages, and DCs;
(ii) co-culturing the isolated T-cells with the APC for at least 30 days, whereby freshly prepared APCs are added for at least 3 times to the original co-culture, and the T-cells proliferate;
(iii) assessing the ability of the proliferating T-cells of (ii) to produce cytokines selected from the group consisting of TNF-α, IFN-γ, GM-CSF, IL-2 in response to the addition of stimulator cells pulsed with anyone of the peptides/proteins (a) to (b) defined above, whereby the stimulator cells are selected from the group of autologous or allogenic immortalized B-cells, DCs, monocytes, and macrophages pulsed with anyone of the peptides/proteins as defined in (a) to (b) above;
(iv) cloning the TNF-α and/or IFN-γ producing T-cells of (iii) by limiting dilution culture in the presence of autologous or allogenic stimulator cells pulsed with anyone of the peptides/proteins (a) to (b) as defined above, and feeder cells, whereby the feeder cells are selected from the group of allogenic or autologous immortalized B-cells, LG2-EBV and allogenic or autologous PBMCs; and
(v) maintaining the isolated T-cell clone of step (iv) in the presence of feeder cells in culture medium comprising a maturation cocktail.

45. The method of claim 44 which is suitable to prepare a CD4⁺ or CD8⁺ T cell clone.

46. The method of claim 44, wherein the maturation cocktail comprises interleukin-2 (IL-2), interleukin-7 (IL-7) and phytohemagglutinin (PHA) 13.

47. A mature DC loaded with one or more of the peptides/proteins selected from the group consisting of (a) one or more of the MCSP peptides and functional variants of claim 24, (b) one or more fusion proteins comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain, and (c) the full length MCSP protein of SEQ ID NO:1 and fragments thereof.

48. A T-cell clone specific for one or more of the peptides/proteins selected from (a) one or more of the MCSP peptides and functional variants of claim 24, (b) one or more fusion proteins comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain, and (c) the full length MCSP protein of SEQ ID NO:1 and fragments thereof.

49. The T-cell clone of claim 48 which is a CD4⁺ or CD8⁺ T cell clone.

50. An antibody specific for the MCSP peptide of claim 24.

51. An antibody specific for the fusion protein of claim 33.

52. A pharmaceutical or diagnostic composition comprising one or more of the functional components selected from the group consisting of: the MCSP peptides and functional variants of claim 24; a fusion protein comprising as a first domain an MCSP peptide according to claim 24 (MCSP domain) and at least one second domain; a nucleic acid sequence encoding the MCSP peptide of claim 24; a nucleic acid sequence encoding a fusion protein comprising as a first domain an MCSP peptide according to claim 24 (MCSP domain) and at least one second domain; a vector comprising a nucleic acid sequence encoding the MCSP peptide of claim 24; a vector comprising a nucleic acid sequence encoding a fusion protein comprising as a first domain an MCSP peptide according to claim 24 (MCSP domain) and at least one second domain; a cell transfected or transformed with a vector comprising a nucleic acid sequence encoding the MCSP peptide of claim 24; a cell transfected or transformed with a vector comprising a nucleic acid sequence encoding a fusion protein comprising as a first domain an MCSP peptide according to claim 24 (MCSP domain) and at least one second domain; a mature DC loaded with one or more of the peptides/proteins selected from the group consisting of (a) one or more of the MCSP peptides and functional variants of claim 24, (b) one or more fusion proteins comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain, and (c) the full length MCSP protein of SEQ ID NO:1 and fragments thereof; a T-cell clone specific for one or more of the peptides/proteins selected from (a) one or more of the MCSP peptides and functional variants of claim 24, (b) one or more fusion proteins comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain, and (c) the full length MCSP protein of SEQ ID NO:1 and fragments thereof; an antibody specific for the MCSP peptide of claim 24; and an antibody specific for a fusion protein comprising as a first domain an MCSP peptide according to claim 24 (MCSP domain) and at least one second domain; and a pharmaceutically or diagnostically acceptable carrier.

53. The composition of claim 52, which is a vaccine further comprising an adjuvant.

54. A method for diagnosing and/or monitoring a disorder characterized by the expression of one or more of the MCSP peptides of claim 24, the full length MCSP protein of SEQ ID NO:1 and fragments thereof, which method comprises the following steps:
contacting a biological sample isolated from a subject having or suspected to have said disorder, with an agent that is specific for anyone of the MCSP peptides of claim 24, the full length MCSP protein of SEQ ID NO:1 and fragments thereof; and determining the interaction between the agent and the peptide.

55. A method for preventing or treating cancer in a patient, which method comprises administering to the patient an effective amount of one or more of the agents selected from the group consisting of: the MCSP peptides and/or the functional variants of claim 24; a fusion protein comprising as a first domain an MCSP peptide according to claim 24 (MCSP domain) and at least one second domain; a nucleic acid sequence encoding the MCSP peptide of claim 24; a nucleic acid sequence encoding a fusion protein comprising as a first domain an MCSP peptide according to claim 24 (MCSP domain) and at least one second domain; a vector comprising a nucleic acid sequence encoding the MCSP peptide of claim 24; a vector comprising a nucleic acid sequence encoding a fusion protein comprising as a first domain an MCSP peptide according to claim 24 (MCSP domain) and at least one second domain, the full length MCSP protein of SEQ ID NO:1 and fragments thereof, nucleic acid sequences encoding the full length MCSP protein of SEQ ID NO:1 or fragments thereof, and vectors comprising a nucleic acid sequence encoding the full length MCSP protein of SEQ ID NO:1 or fragments thereof; a cell transfected or transformed with a vector comprising a nucleic acid sequence encoding the MCSP peptide of claim 24; a cell transfected or transformed with a vector comprising a nucleic acid sequence encoding a fusion protein comprising as a first domain an MCSP peptide according to claim 24 (MCSP domain) and at least one second domain; a mature DC loaded with one or more of the peptides/proteins selected from the group consisting of (a) one or more of the MCSP peptides and functional variants of claim 24, (b) one or more fusion proteins comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain, and (c) the full length MCSP protein of SEQ ID NO:1 and fragments thereof; a T-cell clone specific for one or more of the peptides/proteins selected from (a) one or more of the MCSP peptides and functional variants of claim 24, (b) one or more fusion proteins comprising as a first domain an antigenic T-cell stimulatory peptide (MCSP peptide) which is derived from the melanoma-associated chondroitin sulfate proteoglycan (MCSP), has up to 100 amino acid residues, and comprises at least 8 amino acid residues out of the MCSP segment represented by amino acid residues 644 to 743 of MCSP of SEQ ID NO:1, or a functional variant or salt thereof (MCSP domain), and at least one second domain, and (c) the full length MCSP protein of SEQ ID NO:1 and fragments thereof; an antibody specific for the MCSP peptide of claim 24; and an antibody specific for a fusion protein comprising as a first domain an MCSP peptide according to claim 24 (MCSP domain) and at least one second domain; a pharmaceutical composition comprising an of the above and a pharmaceutical composition comprising any of the above and further comprising an adjuvant.

56. The method of claim 55, wherein the cancer is selected from the group consisting of melanoma, including cutaneous and ocular melanoma, and MCSP expressing tumours, including breast cancer, lobular breast carcinoma, astrocytoma, glioma, glioblastoma, neuroblastoma, sarcoma and certain types of leukaemia.

57. A method for diagnosing cancer which comprises utilizing a diagnostic marker selected from the group consisting of the MCSP peptides or variants of claim 24, a fusion protein comprising as a first domain an MCSP peptide according to claim 24 (MCSP domain) and at least one second domain, a nucleic acid sequence encoding the MCSP peptide of claim 2, a nucleic acid sequence encoding a fusion protein comprising as a first domain an MCSP peptide according to claim 24 (MCSP domain) and at least one second domain, a vector comprising a nucleic acid sequence encoding the MCSP peptide of claim 24, a vector comprising a nucleic acid sequence encoding a fusion protein comprising as a first domain an MCSP peptide according to claim 24 (MCSP domain) and at least one second domain; the full length MCSP protein of SEQ ID NO:1 and fragments thereof, a nucleic acid sequence encoding the full length MCSP protein of SEQ ID NO:1 or fragments thereof, and vectors comprising nucleic acid sequences encoding the full length MCSP protein of SEQ ID NO:1 or fragments thereof.

58. The method of claim 57, wherein the cancer is selected from the group consisting of melanoma, including cutaneous and ocular melanoma, and MCSP expressing tumours including breast cancer, lobular breast carcinoma, astrocytoma, glioma, glioblastoma, neuroblastoma, sarcoma and certain types of leukaemia.

59. A method for inducing a T cell response in a patient, which method comprises administering to the patient an effective amount of one or more of the agents selected from the group consisting of: the MCSP peptides and/or the functional variants of claim 24, a fusion protein comprising as a first domain an MCSP peptide according to claim 24 (MCSP domain) and at least one second domain, a nucleic acid sequence encoding the MCSP peptide of claim 24, a nucleic acid sequence encoding a fusion protein comprising as a first domain an MCSP peptide according to claim 24 (MCSP domain) and at least one second domain; a vector comprising a nucleic acid sequence encoding the MCSP peptide of claim 24, a vector comprising a nucleic acid sequence encoding a fusion protein comprising as a first domain an MCSP peptide according to claim 24 (MCSP domain) and at least one second domain, the full length MCSP protein of SEQ ID NO:1 and fragments thereof, nucleic acid sequences encoding the full length MCSP protein of SEQ ID NO:1 or fragments thereof, and vectors comprising a nucleic acid sequence encoding the full length MCSP protein of SEQ ID NO:1 or fragments thereof.


SEQUENCE LISTING (54 sequences)

SEQ ID NO: 1 - protein, 2322 aa, Homo sapiens [sequence omitted]

SEQ ID NO: 2 - DNA, 7011 bp, Homo sapiens
atgcagtccggccgcggccccccacttccagcccccggcctggccttggctttgaccctgactatgttggccagacttgcatccgcggcttccttcttcggtgagaaccacctggaggtgcctgtggccacggctctgaccgacatagacctgcagctgcagttctccacgtcccagcccgaagccctccttctcctggcagcaggcccagctgaccacctcctgctgcagctctactctggacgcctgcaggtcagacttgttctgggccaggaggagctgaggctgcagactccagcagagacgctgctgagtgactccatcccccacactgtggtgctgactgtcgtagagggctgggccacgttgtcagtcgatgggtttctgaacgcctcctcagcagtcccaggagcccccctagaggtcccctatgggctctttgttgggggcactgggacccttggcctgccctacctgaggggaaccagccgacccctgaggggttgcctccatgcagccaccctcaatggccgcagcctcctccggcctctgacccccgatgtgcatgagggctgtgctgaagagttttctgccagtgatgatgtggccctgggcttctctgggccccactctctggctgccttccctgcctggggcactcaggacgaaggaaccctagagtttacactcaccacacagagccggcaggcacccttggccttccaggcagggggccggcgtggggacttcatctatgtggacatatttgagggccacctgcgggccgtggtggagaagggccagggtaccgtattgctccacaacagtgtgcctgtggccgatgggcagccccatgaggtcagtgtccacatcaatgctcaccggctggaaatctccgtggaccagtaccctacgcatacttcgaaccgaggagtcctcagctacctggagccacggggcagtctccttctcggggggctggatgcagaggcctctcgtcacctccaggaacaccgcctgggcctgacaccagaggccaccaatgcctccctgctgggctgcatggaagacctcagtgtcaatggccagaggcgggggctgcgggaagctttgctgacgcgcaacatggcagccggctgcaggctggaggaggaggagtatgaggacgatgcctatggacattatgaagctttctccaccctggcccctgaggcttggccagccatggagctgcctgagccatgcgtgcctgagccagggctgcctcctgtctttgccaatttcacccagctgctgactatcagcccactggtggtggccgaggggggcacagcctggcttgagtggaggcatgtgcagcccacgctggacctgatggaggctgagctgcgcaaatcccaggtgctgttcagcgtgacccgaggggcacgccatggcgagctcgagctggacatcccgggagcccaggcacgaaaaatgttcaccctcctggacgtggtgaaccgcaaggcccgcttcatccacgatggctctgaggacacctccgaccagctggtgctggaggtgtcggtgacggctcgggtgcccatgccctcatgccttcggaggggccaaacatacctcctgcccatccaggtcaaccctgtcaatgacccaccccacatcatcttcccacatggcagcctcatggtgatcctggaacacacgcagaagccgctggggcctgaggttttccaggcctatgacccggactctgcctgtgagggcctcaccttccaggtccttggcacctcctctggcctccccgtggagcgccgagaccagcctggggagccggcgaccgagttctcctgccgggagttggaggccggcagcctagtctatgtccaccgcggtggtcctgcacaggacttgacgttccgggtcagcgatggactgcaggccagccccccggccacgctgaaggtggtggccatccggccggccatacagatccaccgcagcacagggttgcgactggcccaaggctctgccatgcccatcttgcccgccaacctgtcggtggagaccaatgccgtggggcaggatgtgagcgtgctgttccgcgtcactggggccctgcagtttggggagctgcagaagcagggggcaggtggggtggagggtgctgagtggtgggccacacaggcgttccaccagcgggatgtggagcagggccgcgtgaggtacctgagcactgacccacagcaccacgcttacgacaccgtggagaacctggccctggaggtgcaggtgggccaggagatcctgagcaatctgtccttcccagtgaccatccagagagccactgtgtggatgctgcggctggagccactgcacactcagaacacccagcaggagaccctcaccacagcccacctggaggccaccctggaggaggcaggcccaagccccccaaccttccattatgaggtggttcaggctcccaggaaaggcaaccttcaactacagggcacaaggctgtcagatggccagggcttcacccaggatgacatacaggctggccgggtgacctatggggccacagcacgtgcctcagaggcagtcgaggacaccttccgtttccgtgtcacagctccaccatatttctccccactctataccttccccatccacattggtggtgacccagatgcgcctgtcctcaccaatgtcctcctcgtggtgcctgagggtggtgagggtgtcctctctgctgaccacctctttgtcaagagtctcaacagtgccagctacctctatgaggtcatggagcggccccgccatgggaggttggcttggcgtgggacacaggacaagaccactatggtgacatccttcaccaatgaagacctgttgcgtggccggctggtctaccagcatgatgactccgagaccacagaagatgatatcccatttgttgctacccgccagggcgagagcagtggtgacatggcctgggaggaggtacggggtgtcttccgagtggccatccagcccgtgaatgaccacgcccctgtgcagaccatcagccggatcttccatgtggcccggggtgggcggcggctgctgactacagacgacgtggccttcagcgatgctgactcgggctttgctgacgcccagctggtgcttacccgcaaggacctcctctttggcagtatcgtggccgtagatgagcccacgcggcccatctaccgcttcacccaggaggacctcaggaagaggcgagtactgttcgtgcactcaggggctgaccgtggctggatccagctgcaggtgtccgacgggcaacaccaggccactgcgctgctggaggtgcaggcctcggaaccctacctccgtgtggccaacggctccagccttgtggtccctcaagggggccagggcaccatcgacacggccgtgctccacctggacaccaacctcgacatccgcagtggggatgaggtccactaccacgtcacagctggccctcgctggggacagctagtccgggctggtcagccagccacagccttctcccagcaggacctgctggatggggccgttctctatagccacaatggcagcctcagcccccgcgacaccatggccttctccgtggaagcagggccagtgcacacggatgccaccctacaagtgaccattgccctagagggcccactggccccactgaagctggtccggcacaagaagatctacgtcttccagggagaggcagctgagatcagaagggaccagctggaggcagcccaggaggcagtgccacctgcagacatcgtattctcagtgaagagcccaccgagtgccggctacctggtgatggtgtcgcgtggcgccttggcagatgagccacccagcctggaccctgtgcagagcttctcccaggaggcagtggacacaggcagggtcctgtacctgcactcccgccctgaggcctggagcgatgccttctcgctggatgtggcctcaggcctgggtgctcccctcgagggcgtccttgtggagctggaggtgctgcccgctgccatcccactagaggcgcaaaacttcagcgtccctgagggtggcagcctcaccctggcccctccactgctccgtgtctccgggccctacttccccactctcctgggcctcagcctgcaggtgctggagccaccccagcatggagccctgcagaaggaggacggacctcaagccaggaccctcagcgccttctcctggagaatggtggaagagcagctgatccgctacgtgcatgacgggagcgagacactgacagacagttttgtcctgatggctaatgcctccgagatggatcgccagagccatcctgtggccttcactgtcactgtcctgcctgtcaatgaccaaccccccatcctcactacaaacacaggcctgcagatgtgggagggggccactgcgcccatccctgcggaggctctgaggagcacggacggcgactctgggtctgaggatctggtctacaccatcgagcagcccagcaacgggcgggtagtgctgcggggggcgccgggcactgaggtgcgcagcttcacgcaggcccagctggacggcgggctcgtgctgttctcacacagaggaaccctggatggaggcttccgcttccgcctctctgacggcgagcacacttcccccggacacttcttccgagtgacggcccagaagcaagtgctcctctcgctgaagggcagccagacactgactgtctgcccagggtccgtccagccactcagcagtcagaccctcagggccagctccagcgcaggcactgacccccagctcctgctctaccgtgtggtgcggggcccccagctaggccggctgttccacgcccagcaggacagcacaggggaggccctggtgaacttcactcaggcagaggtctacgctgggaatattctgtatgagcatgagatgccccccgagcccttttgggaggcccatgataccctagagctccagctgtcctcgccgcctgcccgggacgtggccgccacccttgctgtggctgtgtcttttgaggctgcctgtccccagcgccccagccacctctggaagaacaaaggtctctgggtccccgagggccagcgggccaggatcaccgtggctgctctggatgcctccaatctcttggccagcgttccatcaccccagcgctcagagcatgatgtgctcttccaggtcacacagttccccagccggggccagctgttggtgtccgaggagcccctccatgctgggcagccccacttcctgcagtcccagctggctgcagggcagctagtgtatgcccacggcggtgggggcacccagcaggatggcttccactttcgtgcccacctccaggggccagcaggggcctccgtggctggaccccaaacctcagaggcctttgccatcacggtgagggatgtaaatgagcggccccctcagccacaggcctctgtcccactccggctcacccgaggctctcgtgcccccatctcccgggcccagctgagtgtggtggacccagactcagctcctggggagattgagtacgaggtccagcgggcaccccacaacggcttcctcagcctggtgggtggtggcctggggcccgtgacccgcttcacgcaagccgatgtggattcagggcggctggccttcgtggccaacgggagcagcgtggcaggcatcttccagctgagcatgtctgatggggccagcccacccctgcccatgtccctggctgtggacatcctaccatccgccatcgaggtgcagctgcgggcacccctggaggtgccccaagctttggggcgctcctcactgagccagcagcagctccgggtggtttcagatcgggaggagccagaggcagcataccgcctcatccagggaccccagtatgggcatctcctggtgggcgggcggcccacctcggccttcagccaattccagatagaccagggcgaggtggtctttgccttcaccaacttctcctcctctcatgaccacttcagagtcctggcactggctaggggtgtcaatgcatcagccgtagtgaacgtcactgtgagggctctgctgcatgtgtgggcaggtgggccatggccccagggtgccaccctgcgcctggaccccaccgtcctagatgctggcgagctggccaaccgcacaggcagtgtgccgcgcttccgcctcctggagggaccccggcatggccgcgtggtccgcgtgccccgagccaggacggagcccgggggcagccagctggtggagcagttcactcagcaggaccttgaggacgggaggctggggctggaggtgggcaggccagaggggagggcccccggccccgcaggtgacagtctcactctggagctgtgggcacagggcgtcccgcctgctgtggcctccctggactttgccactgagccttacaatgctgcccggccctacagcgtggccctgctcagtgtccccgaggccgcccggacggaagcagggaagccagagagcagcacccccacaggcgagccaggccccatggcatccagccctgagcccgctgtggccaagggaggcttcctgagcttccttgaggccaacatgttcagcgtcatcatccccatgtgcctggtacttctgctcctggcgctcatcctgcccctgctcttctacctccgaaaacgcaacaagacgggcaagcatgacgtccaggtcctgactgccaagccccgcaacggcctggctggtgacaccgagacctttcgcaaggtggagccaggccaggccatcccgctcacagctgtgcctggccaggggccccctccaggaggccagcctgacccagagctgctgcagttctgccggacacccaaccctgcccttaagaatggccagtactgggtgtgaggcctggcctgggcccagatgctgatcgggccagggacaggc

SEQ ID NO: 3 - protein, 42 aa, Artificial Sequence
  Description of Artificial Sequnece T-cell stimulatory peptide
LAQGSAMPILPANLSVETNAVGQDVSVLFRVTGALQFGELQK

SEQ ID NO: 4 - protein, 16 aa, Artificial Sequence
  Description of Artificial Sequnece T-cell stimulatory peptide
LAQGSAMPILPANLSV

SEQ ID NO: 5 - protein, 16 aa, Artificial Sequence
  Description of Artificial Sequnece T-cell stimulatory peptide
SAMPILPANLSVETNA

SEQ ID NO: 6 - protein, 16 aa, Artificial Sequence
  Description of Artificial Sequnece T-cell stimulatory peptide
ILPANLSVETNAVGQD

SEQ ID NO: 7 - protein, 16 aa, Artificial Sequence
  Description of Artificial Sequnece T-cell stimulatory peptide
NLSVETNAVGQDVSVL

SEQ ID NO: 8 - protein, 16 aa, Artificial Sequence
  Description of Artificial Sequnece T-cell stimulatory peptide
ETNAVGQDVSVLFRVT

SEQ ID NO: 9 - protein, 16 aa, Artificial Sequence
  Description of Artificial Sequnece T-cell stimulatory peptide
VGQDVSVLFRVTGALQ

SEQ ID NO: 10 - protein, 16 aa, Artificial Sequence
  Description of Artificial Sequnece T-cell stimulatory peptide
VSVLFRVTGALQFGEL

SEQ ID NO: 11 - protein, 14 aa, Artificial Sequence
  Description of Artificial Sequnece T-cell stimulatory peptide
FRVTGALQFGELQK

SEQ ID NO: 12 - protein, 16 aa, Artificial Sequence
  Description of Artificial Sequence Peptide derived from MCSP
SQVLFSVTRGAHYGEL

SEQ ID NO: 13 - protein, 16 aa, Artificial Sequence
  Description of Artificial Sequence Peptide derived from MCSP
VRYLSTDPQHHAYDTV

SEQ ID NO: 14 - protein, 16 aa, Artificial Sequence
  Description of Artificial Sequence Peptide derived from MCSP
GEALVNFTQAEVYAGN

SEQ ID NO: 15 - protein, 16 aa, Artificial Sequence
  Description of Artificial Sequence Peptide derived from MCSP
PHEVSVHINAHRLEIS

SEQ ID NO: 16 - DNA, 27 bp, Artificial Sequence
  Description of Artificial Sequence forward primer for MCSP
ggggatcccatccggccggccatacag

SEQ ID NO: 17 - DNA, 31 bp, Artificial Sequence
  Description of Artificial Sequence reverse primer for MCSP
ggggatcctcaccgctggtggaacgcctgtg

SEQ ID NO: 18 - DNA, 23 bp, Artificial Sequence
  Description of Artificial Sequence sense primer for hu-Ii
tttccatggatgaccagcgcgac

SEQ ID NO: 19 - DNA, 30 bp, Artificial Sequence
  Description of Artificial Sequence antisense primer for hu-Ii
tttggatccggaagcttcatgcgcaggttc

SEQ ID NO: 20 - DNA, 474 bp, Artificial Sequence
  Description of Artificial Sequence DNA sequence showing the hu-Ii- MCSP fusion
atggaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaaggatcccatccggccggccatacagatccaccgcagcacagggttgcgactggcccaaggctctgccatgcccatcttgcccgccaacctgtcggtggagaccaatgccgtggggcaggatgtgagcgtgctgttccgcgtcactggggccctgcagtttggggagctgcagaagcagggggcaggtggggtggagggtgctgagtggtgggccacacaggcgttccaccagcggtga

SEQ ID NO: 21 - protein, 158 aa, Artificial Sequence
  Description of Artificial Sequence hu-Ii- MCSP fusion peptide
MDLISNNEQLPMLGRRPGAPESKCSRGALYTGFSILVTLLLAGQATTAYFLYQQQGRLDKLTVTSQNLQLENLRMKLPKDPIRPAIQIHRSTGLRLAQGSAMPILPANLSVETNAVGQDVSVLFRVTGALQFGELQKQGAGGVEGAEWWATQAFHQRZ

SEQ ID NO: 22 - protein, 9 aa, Artificial Sequence
  Description of Artificial Sequence truncated peptide
LFRVTGALQ

SEQ ID NO: 23 - protein, 10 aa, Artificial Sequence
  Description of Artificial Sequence truncated peptide
VLFRVTGALQ

SEQ ID NO: 24 - protein, 11 aa, Artificial Sequence
  Description of Artificial Sequence truncated peptide
SVLFRVTGALQ

SEQ ID NO: 25 - protein, 12 aa, Artificial Sequence
  Description of Artificial Sequence truncated peptide
VSVLFRVTGALQ

SEQ ID NO: 26 - protein, 13 aa, Artificial Sequence
  Description of Artificial Sequence truncated peptide
DVSVLFRVTGALQ

SEQ ID NO: 27 - protein, 14 aa, Artificial Sequence
  Description of Artificial Sequence truncated peptide
QDVSVLFRVTGALQ

SEQ ID NO: 28 - protein, 15 aa, Artificial Sequence
  Description of Artificial Sequence truncated peptide
GQDVSVLFRVTGALQ

SEQ ID NO: 29 - protein, 15 aa, Artificial Sequence
  Description of Artificial Sequence truncated peptide
VGQDVSVLFRVTGAL

SEQ ID NO: 30 - protein, 14 aa, Artificial Sequence
  Description of Artificial Sequence truncated peptide
VGQDVSVLFRVTGA

SEQ ID NO: 31 - protein, 13 aa, Artificial Sequence
  Description of Artificial Sequence truncated peptide
VGQDVSVLFRVTG

SEQ ID NO: 32 - protein, 12 aa, Artificial Sequence
  Description of Artificial Sequence truncated peptide
VGQDVSVLFRVT

SEQ ID NO: 33 - protein, 11 aa, Artificial Sequence
  Description of Artificial Sequence truncated peptide
VGQDVSVLFRV

SEQ ID NO: 34 - protein, 10 aa, Artificial Sequence
  Description of Artificial Sequence truncated peptide
VGQDVSVLFR

SEQ ID NO: 35 - protein, 9 aa, Artificial Sequence
  Description of Artificial Sequence truncated peptide
VGQDVSVLF

SEQ ID NO: 36 - protein, 31 aa, Artificial Sequence
  Description of Artificial Sequence fragment of MCSP
PPADIVFSVKSPPSAGYLVMVSRGALADEPP

SEQ ID NO: 37 - protein, 11 aa, Artificial Sequence
  Description of Artificial Sequence synthetic construct
PPSAGYLVMVS

SEQ ID NO: 38 - protein, 12 aa, Artificial Sequence
  Description of Artificial Sequence fragment of MCSP
PPSAGYLVMVSR

SEQ ID NO: 39 - protein, 13 aa, Artificial Sequence
  Description of Artificial Sequence fragment of MCSP
PPSAGYLVMVSRG

SEQ ID NO: 40 - protein, 14 aa, Artificial Sequence
  Description of Artificial Sequence fragment of MCSP
PPSAGYLVMVSRGA

SEQ ID NO: 41 - protein, 15 aa, Artificial Sequence
  Description of Artificial Sequence fragment of MCSP
PPSAGYLVMVSRGAL

SEQ ID NO: 42 - protein, 16 aa, Artificial Sequence
  Description of Artificial Sequence fragment of MCSP
PPSAGYLVMVSRGALA

SEQ ID NO: 43 - protein, 15 aa, Artificial Sequence
  Description of Artificial Sequence fragment of MCSP
PSAGYLVMVSRGALA

SEQ ID NO: 44 - protein, 14 aa, Artificial Sequence
  Description of Artificial Sequence fragment of MCSP
SAGYLVMVSRGALA

SEQ ID NO: 45 - protein, 13 aa, Artificial Sequence
  Description of Artificial Sequence fragment of MCSP
AGYLVMVSRGALA

SEQ ID NO: 46 - protein, 12 aa, Artificial Sequence
  Description of Artificial Sequence fragment of MCSP
GYLVMVSRGALA

SEQ ID NO: 47 - protein, 11 aa, Artificial Sequence
  Description of Artificial Sequence fragment of MCSP
YLVMVSRGALA

SEQ ID NO: 48 - protein, 16 aa, Artificial Sequence
  Description of Artificial Sequence fragment of MCSP
PPADIVFSVKSPPSAG

SEQ ID NO: 49 - protein, 15 aa, Artificial Sequence
  Description of Artificial Sequence fragment of MCSP
IVFSVKSPPSAGYLV

SEQ ID NO: 50 - protein, 16 aa, Artificial Sequence
  Description of Artificial Sequence fragment of MCSP
SVKSPPSAGYLVMVSR

SEQ ID NO: 51 - protein, 16 aa, Artificial Sequence
  Description of Artificial Sequence fragment of MCSP
GYLVMVSRGALADEPP

SEQ ID NO: 52 - protein, 11 aa, Artificial Sequence
  Description of Artificial Sequence fragment of MCSP
YGRKKRRQRRR

SEQ ID NO: 53 - protein, 11 aa, Artificial Sequence
  Description of Artificial Sequence HIV
YARAAARQARA

SEQ ID NO: 54 - protein, 42 aa, Artificial Sequence
  Description of Artificial Sequence fusion peptide
YARAAARQARAPPADIVFSVKSPPSAGYLVMVSRGALADEPP